(19) United States
(12) Patent Application Publication
(10) Pub. No.: US 2026/0078434 A1
(43) Pub. Date: Mar. 19, 2026

(54) DEVICE FOR PARALLEL DETECTION AND QUANTIFICATION OF NUCLEIC ACID BASED MARKERS

(71) Applicant: TODX LTD., Tel Aviv (IL)

(72) Inventors: Noam Kedem Mamet, Tel Aviv (IL); Gil Harari, Tel Aviv (IL)

(21) Appl. No.: 19/109,939

(22) PCT Filed: Sep. 8, 2023
(86) PCT No.: PCT/IB23/00531

§ 371 Date: Mar. 7, 2025

Related U.S. Application Data
(60) Provisional application No. 63/405,224, filed on Sep. 9, 2022.

(51) Int. Cl. C12Q 1/6837; C12Q 1/6809; C12Q 1/6851; C12Q 1/6876

(52) U.S. Cl. CPC C12Q 1/6837; C12Q 1/6809; C12Q 1/6851; C12Q 1/6876; C12Q 2600/16

(57) ABSTRACT

The present disclosure describes methods and devices for detection and quantification of nucleic acid based markers in a sample.

### RELATED APPLICATIONS

This application claims the benefit of priority to U.S. Provisional Patent Application Ser. No. 63/405,224, filed Sep. 9, 2022, which is hereby incorporated by reference in its entirety.

### BACKGROUND

Molecular diagnosis of genetic defects and diseases requires techniques that are capable of detecting small quantities of DNA and/or RNA in a sample, or techniques that are extremely sensitive to detect mutations in such nucleic acids. For example, techniques such as southern blot, polymerase chain reaction (PCR), reverse transcriptase-polymerase chain reaction, and ligase chain reaction have been extensively used to detect microbial and viral pathogens, and to diagnose cancers and genetic diseases.

The development of simple, fast and reliable amplification-based assays, and devices that utilize such assays to detect target nucleic acids will greatly aid molecular diagnosis.

### SUMMARY

Provided herein are methods and devices related to detection and/or quantification of one or more target polynucleotides in a sample.

In some aspects, provided herein is a device comprising a first external surface, a second external surface that is different from the first external surface, an internal surface, a plurality of targeting nucleic acids (e.g., primers) immobilized to pre-determined locations on the internal surface, and a reaction chamber enclosed by the internal surface, wherein targeting nucleic acids specific for different target polynucleotides are immobilized at different predetermined locations on the internal surface.

In some embodiments, the reaction chamber is substantially flat. In some embodiments, the reaction chamber has a surface/volume ratio larger than 1.3/mm, e.g., larger than 1.4/mm, 1.5/mm, 1.6/mm, 1.7/mm, 1.8/mm, 1.9/mm, 2.0/mm, 3.0/mm, 4.0/mm, 5.0/mm, 6.0/mm, 7.0/mm, 8.0/mm, 9.0/mm, 10/mm, 15/mm, 20/mm, 25/mm, 30/mm, 35/mm, 40/mm, 45/mm, 50/mm, 55/mm, 60/mm, 65/mm, 70/mm, 75/mm, 80/mm, 85/mm, 90/mm, 95/mm, or 100/mm. In some embodiments, the reaction chamber has a height of 10-700 microns. In some embodiments, the first external surface comprises a material that is able to conduct heat. In some embodiments, the second external surface is on the opposite side to the first external surface. In some embodiments, the second external surface comprise an insulating material. In some embodiments, at least one external surface is transparent. In some embodiments, the device further comprises a strap or a tape to fix the device to a human body.

In some embodiments, the targeting nucleic acid comprises a primer. In some embodiments, only forward primers or reverse primers specific to the target polynucleotides are immobilized to the internal surface. In some embodiments, the reaction chamber comprises the other primer that forms a primer pair with the immobilized forward primer or reverse primer. In some embodiments, both forward and reverse primers specific to the target polynucleotides are immobilized to the internal surface. In some embodiments, the primers comprise a set of nested primers for the same target polynucleotide.

In some embodiments, the targeting nucleic acids specific for each target polynucleotide are uniformly immobilized to the predetermined location of the internal surface. In some embodiments, the plurality of targeting nucleic acids are immobilized as an array of targeting nucleic acid clusters, with each targeting nucleic acid cluster located at one of the predetermined locations on the internal surface. In some embodiments, the targeting nucleic acid clusters are spatially separated from each other in space on the internal surface. In some embodiments, multiple clusters of targeting nucleic acids for the same target polynucleotides are immobilized on the internal surface. In some embodiments, one cluster of targeting nucleic acids spreads into another cluster of targeting nucleic acids within the set of nested targeting nucleic acids for the target polynucleotides. In some embodiments, each cluster of targeting nucleic acids comprises at least 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or more copies of the same targeting nucleic acids or set of targeting nucleic acids specific for the same target polynucleotide. In some embodiments, the targeting nucleic acid cluster is immobilized to a surface area with a size at least 0.01 mm², 0.0198 mm², 0.02 mm², 0.03 mm², 0.04 mm², 0.05 mm², 0.06 mm², 0.07 mm², 0.08 mm², 0.09 mm², 0.1 mm², 0.2 mm², 0.3 mm², 0.4 mm², 0.5 mm², 0.6 mm², 0.7 mm², 0.8 mm², 0.9 mm², 1 mm², 2 mm², 3 mm², 4 mm², 5 mm², 6 mm², 7 mm², 8 mm², 9 mm², or 10 mm².

In some embodiments, the targeting nucleic acid is immobilized to the internal surface via 5′ end. In some embodiments, the targeting nucleic acid is covalently linked to the internal surface. In some embodiments, the targeting nucleic acid is 5′DBCO modified and the internal surface is azide functionalized surface. In some embodiments, the targeting nucleic acid is 5′amine modified and the internal surface is NHS functionalized surface. In some embodiments, the targeting nucleic acid is non-covalently linked to the internal surface. In some embodiments, the targeting nucleic acid is immobilized to the internal surface via passive absorption, streptavidin-biotin interaction, or hybridization. In some embodiments, the 3′end of the targeting nucleic acid is free to elongate. In some embodiments, the targeting nucleic acids are immobilized on a 2-dimensional surface. In some such embodiments, each cluster comprises targeting nucleic acids at a density of at least 9/micron², e.g., at least 10/micron², 15/micron², 20/micron², 25/micron², 30/micron², 35/micron², 40/micron², 45/micron², 50/micron², 55/micron², 60/micron², 65/micron², 70/micron², 75/micron², 80/micron², 85/micron², 90/micron², 95/micron², 10²/micron², 5×10²/micron², 10³/micron², 5×10³/micron², 10⁴/micron², 5×10⁴/micron², or 10⁵/micron². In some embodiments, the targeting nucleic acids are immobilized on a 3-dimensional surface. In some such embodiments, each cluster comprises targeting nucleic acids at a density of at least 9/micron³, e.g., at least 10/micron³, 15/micron³, 20/micron³, 25/micron³, 30/micron³, 35/micron³, 40/micron³, 45/micron³, 50/micron³, 55/micron³, 60/micron³, 65/micron³, 70/micron³, 75/micron³, 80/micron³, 85/micron³, 90/micron³, 95/micron³, 10²/micron³, 5×10²/micron³, 10³/micron³, 5×10³/micron³, 10⁴/micron³, or 5×10⁴/micron³, 10⁵/micron³, 5×10⁵/micron³, 10⁶/micron³, or 5×10⁶/micron³. In some embodiments, the targeting nucleic acid is an invading primer. In some embodiments, the invading primer comprises Locked Nucleic Acid (LNA), Peptide Nucleic Acid (PNA), Phosphorothioates (PTO), Zip Nucleic Acids (ZNA), invader probe, or Intercalating Nucleic Acid (INA).

In some embodiments, the device further comprises a reaction mix in the reaction chamber. In some embodiments, the reaction mix comprises dNTPs, buffer, and at least one enzyme for isothermal amplification. In some embodiments, the isothermal amplification is Transcription Mediated Amplification (TMA), Nucleic Acid Sequence Based Amplification (NASBA), Loop Mediated Isothermal Amplification (LAMP), Hinge Initiated Primer Dependent Amplification (HIP), Helicase Dependent Amplification (HDA), Recombinase Polymerase Amplification (RPA), Strand Displacement Amplification (SDA), or rolling circle amplification.

In some embodiments, the device is used for detecting presence of one or more target polynucleotides in a sample. In some embodiments, the device is used for quantifying the amount of one or more target polynucleotides in a sample. In some embodiments, the device is used at a body temperature. In some embodiments, one or more target polynucleotides are detected using the naked eye or a cell phone camera. In some embodiments, the sample comprises a reference sequence and targeting nucleic acids for the reference sequence are immobilized to the inner surface. In some embodiments, the targeting nucleic acids are positioned in a specific pattern according to a test serial number. In some embodiments, the internal surface is a 3D polymer.

In some embodiments, the device further comprises a sample container, a reagent compartment, and a waste reservoir, each of which is connected to the reaction chamber. In some embodiments, the device further comprises one or more valves selected from: (1) a first valve which controls fluid flow from the sample container to the reaction chamber; (2) a second valve which controls fluid flow from the reagent compartment to the reaction chamber.; and (3) a third valve which controls fluid flow from the reaction chamber to the waste reservoir.

In some embodiments, the sample container contains lysis buffer. In some embodiments, the sample container has a proximal end that is connected to the reaction chamber and an open distal end that is configured to receive a raw sample from a carrier. In some embodiments, the sample container is configured to receive a raw sample carried by a swab having a proximal end and a distal end. In some embodiments, the lysis buffer can be in fluid communication with the raw sample to form a lysed sample which dispenses into the reaction chamber.

In some embodiments, the reagent compartment and/or the sample container is disposed upstream of the waste reservoir. In some embodiments, the reagent compartment is disposed upstream of the lysed sample entry point. In some embodiments, the reagent compartment, the reaction chamber, and the waste reservoir are horizontally aligned. In some embodiments, the sample container is positioned on top of the reaction chamber at an angle with respect to the reaction chamber. In some embodiments, the angle is about 90 degree.

In some embodiments, the reagent compartment comprises a first reagent chamber and a second reagent chamber, and a fourth valve; and wherein the fourth valve controls fluid flow from the first reagent chamber to the second reagent chamber. In some embodiments, the first reagent chamber contains reaction buffer, and the second reagent chamber contains dried enzymes, and wherein the reaction buffer dispenses into the second reagent chamber to dissolve the dried enzymes to form a reaction mix which then dispenses into the reaction chamber. In some embodiments, the second reagent chamber is disposed downstream of the first reagent chamber and upstream of the reaction chamber. In some embodiments, the first reagent chamber, the second reagent chamber, and the reaction chamber are horizontally aligned. In some embodiments, the second valve controlling fluid flow from the reagent compartment to the reaction chamber controls the fluid flow from the second reagent chamber to the reaction chamber.

In some embodiments, the second valve controlling fluid flow from the reagent compartment to the reaction chamber is a timed valve. In some embodiments, the timed valve is configured to open after a period of time after the first valve and the fourth valve are open. In some embodiments, the timed valve is a dissolvable membrane. In some embodiments, the membrane is made of a material that can be dissolved by the reaction buffer. In some embodiments, the membrane is completely dissolved by the reaction buffer after a period of time after the first valve and the fourth valve are open. In some embodiments, the period of time is calibrated by the width of the membrane. In some embodiments, the timed valve is a mechanical gate that is timed and controlled by pressure building, springs, electronic timer, Bluetooth signal from a phone application.

In some embodiments, the reaction chamber comprises a plurality of internal baffles defining a nonlinear flow path from the lysed sample entry point and/or the reaction mix entry point to the waste reservoir.

In some embodiments, the sample container is configured to open the first valve when it is being detached, thereby allowing the lysed sample to flow into the reaction chamber before the sample container is detached. In some embodiments, the sample container is configured to open the fourth valve when it is being detached. In some embodiments, the device further comprises a screw cap that can be screwed to the distal end of the sample container to seal it. In some embodiments, the screw cap is attached to the distal end of the swab on one side and to a swab handle on the opposite side such that the swab can be placed into the sample container with the proximal end immersed in the lysis buffer when the screw cap is screwed onto the sample container. In some embodiments, the device is configured in a way that continued motion of rotating the swab handle in the same direction after the screw cap is fully screwed opens the first valve and the fourth valve and then detaches the sample container. In some embodiments, the device is configured in a way that shoving the swab into the sample container opens the first valve and the fourth valve.

In some embodiments, the sample container comprises a plurality of undulating interior sidewalls with peaks and valleys. In some embodiments, each peak of the interior sidewalls has the same dimensions. In some embodiments, the interior sidewalls are configured in a way that the swab is squeezed at each peak of the interior sidewalls. In some embodiments, all peaks of the interior sidewalls are immersed in the lysis buffer.

In some embodiments, the swab is configured to allow fluid flowing through the proximal end of the swab. In some embodiments, the swab comprises a pipe and a plunger, and wherein the plunger is connected to the pipe at one end and to the screw cap at the opposite end.

In some embodiments, the end of the pipe that is opposite to the end connected to the plunger is coated with a wad of fibers and comprises a plurality of fluid outlet holes. In some embodiments, the pipe comprises a fluid inlet hole with one directional valve above the wad of fibers. In some embodiments, the pipe, near the end that is connected to the plunger, comprises an air hole which is configured to let air out when the lysis buffer enters into the pipe via the fluid inlet hole and to be sealed when the plunger is pushed down.

In some embodiments, a QR code is pre-printed on the device. In some embodiments, the QR code comprises data representing a test type, a serial or batch ID, a pattern ID, and an expiration date. In some embodiments, the device further comprises a visual fiducial.

In certain aspects, provided herein is a method of quantifying one or more target polynucleotides in a sample, the method comprising: (a) providing a device described herein; (b) dispensing a sample to the reaction chamber; (c) incubating the device at a temperature for a period of time such that the presence of a target polynucleotide in the sample results in the generation of a cluster of immobilized amplicons on the internal surface of the reaction chamber at the predetermined location at which the targeting nucleic acid specific for that target polynucleotide was immobilized; and (d) counting the number of distinct clusters of amplicons for each target polynucleotide at the pre-determined location to quantify the amount of each target polynucleotide in the sample.

In certain aspects, provided herein is a method of detecting the presence of one or more target polynucleotides in a sample (if any are present), the method comprising: (a) providing a device described herein; (b) dispensing a sample to the reaction chamber; (c) incubating the device at a temperature for a period of time such that the presence of a target polynucleotide in the sample results in the generation of a cluster of immobilized amplicons on the internal surface of the reaction chamber at the predetermined location at which the targeting nucleic acid specific for that target polynucleotide was immobilized; and (d) detecting the position of clusters of amplicons on the solid surface to detect the presence of one or more target polynucleotides in the sample.

In some embodiments, the temperature is an ambient temperature. In some embodiments, the temperature is about 37° C. to about 42° C. In some embodiments, the device is incubated on human body. In some embodiments, the method further comprises dispensing a reaction mix to the reaction chamber. In some embodiments, the clusters of amplicons are detected using the naked eye or a cell phone camera. It is to be understood that both the foregoing general description and the following detailed description are exemplary and are intended to provide further explanation of the disclosed subject matter claimed.

The accompanying drawings, which are incorporated in and constitute part of this specification, are included to illustrate and provide a further understanding of the method and system of the disclosed subject matter. Together with the description, the drawings serve to explain the principles of the disclosed subject matter.

### BRIEF DESCRIPTION OF FIGURES

A detailed description of various aspects, features, and embodiments of the subject matter described herein is provided with reference to the accompanying drawings, which are briefly described below. The drawings are illustrative and are not necessarily drawn to scale, with some components and features being exaggerated for clarity. The drawings illustrate various aspects and features of the present subject matter and may illustrate one or more embodiment(s) or example(s) of the present subject matter in whole or in part.

FIG. 1 is a schematic diagram of a set of spatially separated clusters positioned on a solid surface.

FIGS. 2-6 are schematic representations of certain exemplary embodiments provided herein.

FIG. 7 is an exemplary digital PCR (dPCR) flow chart.

FIG. 8 shows schematic illustration of targeting nucleic acid spotting.

FIGS. 9A-9E show schematic illustration of UMI generation.

FIG. 10 shows schematic illustration of UMI amplification.

FIGS. 11A-11D show schematic illustration of rapid blocking of a cluster using Hinge Initiated Primer dependent amplification (HIP) following first release of UMI from said cluster.

FIG. 12 is a schematic representation of an exemplary device with a timed membrane, in accordance with the disclosed subject matter.

FIG. 13 is a schematic representation of an exemplary reaction chamber with a plurality of internal baffles defining a nonlinear flow path, in accordance with the disclosed subject matter.

FIG. 14 is a schematic representation of an exemplary device with two gates: one controls fluid flow from the sample tube to the reaction chamber and the other controls fluid flow from the chamber with reaction buffer to the chamber with dried enzymes, in accordance with the disclosed subject matter.

FIGS. 15A-15C are schematic representations of exemplary devices with a screw cap connected to a swab handle, in accordance with the disclosed subject matter.

FIGS. 16A-16B are schematic representations of exemplary sample tubes that have undulating sidewalls with peaks and valleys, in accordance with the disclosed subject matter.

FIG. 17 is a schematic representation of an exemplary swab with: 1—a wad of cotton fibers and many tight holes at the tip of the swab; 2—a buffer inlet with one directional valve just above the wad of cotton fibers; and 3—an air hole at the other end of the swab just under the plunger, in accordance with the disclosed subject matter.

### DETAILED DESCRIPTION

### General

Provided herein are methods and devices related to the detection and/or quantification of one or more target polynucleotides in a sample.

Definitions The articles “a” and “an” are used herein to refer to one or to more than one (e.g., to at least one) of the grammatical object of the article. By way of example, “an element” means one element or more than one element.

As used herein, the term “targeting nucleic acid” refers to an immobilized nucleic acid molecule able to specifically bind to a target polynucleotide and/or assist in its amplification, detection, or quantification. In some embodiments, the targeting nucleic acid comprises a primer. In some embodiments, the targeting nucleic acid comprises one or more of a linker, a UMI, a promoter (e.g., T7 promoter), a secondary structure forming stretch (e.g., a 3′primer blocker, hinge, etc.), a probe, etc. In some embodiments, the targeting nucleic acid has a length that is less than 100 bases, e.g., less than 95, 90, 85, 80, 75, 70, 65, 60, 55, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5 bases.

As used herein, the term “targeting nucleic acid cluster” refers to a spatially separate set of locally immobilized targeting nucleic acids (e.g., primers) designated specifically for (e.g., binding specifically to) a particular target polynucleotide.

The term “binding” or “interacting” refers to an association, which may be a stable association, between two molecules, e.g., between a targeting nucleic acid and target, e.g., due to, for example, electrostatic, hydrophobic, ionic and/or hydrogen-bond interactions.

As used herein, two nucleic acid sequences “complement” one another or are “complementary” to one another if they base pair one another at each position.

As used herein, two or more nucleic acid sequences “correspond” to each other if they are all complementary to the same nucleic acid sequence.

As used herein, “specific binding” refers to the ability of a targeting nucleic acid to bind to a predetermined target. Typically, a targeting nucleic acid specifically binds to its target with an affinity corresponding to a K_{D} of about 10⁻⁷ M or less, about 10⁻⁸ M or less, or about 10⁻⁹ M or less and binds to the target with a K_{D} that is significantly less (e.g., at least 2 fold less, at least 5 fold less, at least 10 fold less, at least 50 fold less, at least 100 fold less, at least 500 fold less, or at least 1000 fold less) than its affinity for binding to a non-specific and unrelated target.

As used herein, the Tm or melting temperature of two oligonucleotides is the temperature at which 50% of the oligonucleotide/targets are bound and 50% of the oligonucleotide target molecules are not bound. Tm values of two oligonucleotides are oligonucleotide concentration dependent and are affected by the concentration of monovalent, divalent cations in a reaction mixture. Tm can be determined empirically or calculated using the nearest neighbor formula, as described in Santa Lucia, J. PNAS(USA) 95:1460-1465 (1998), which is hereby incorporated by reference.

The terms “polynucleotide” and “nucleic acid” are used herein interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

### Devices

In some aspects, provided herein is a device comprising a first external surface, a second external surface that is different from the first external surface, an internal surface, a plurality of targeting nucleic acids (e.g., primers) immobilized to pre-determined locations on the internal surface, and a reaction chamber enclosed by the internal surface, wherein targeting nucleic acids specific for different target polynucleotides are immobilized at different predetermined locations on the internal surface.

In some embodiments, the reaction chamber is substantially flat. In some embodiments, the reaction chamber has a surface/volume ratio larger than 1.3/mm, e.g., larger than 1.4/mm, 1.5/mm, 1.6/mm, 1.7/mm, 1.8/mm, 1.9/mm, 2.0/mm, 3.0/mm, 4.0/mm, 5.0/mm, 6.0/mm, 7.0/mm, 8.0/mm, 9.0/mm, 10/mm, 15/mm, 20/mm, 25/mm, 30/mm, 35/mm, 40/mm, 45/mm, 50/mm, 55/mm, 60/mm, 65/mm, 70/mm, 75/mm, 80/mm, 85/mm, 90/mm, 95/mm, or 100/mm.

In some embodiments, the first external surface comprises a material that is able to conduct heat. For example, the material for the first external surface can be selected from thermally conductive material known in the art, for example, a metal (e.g., copper, brass, aluminum, a metal alloy), beryllium oxide ceramics, or a thermally conductive plastic/polymer. Preferably, the external surface of the device that is closest to the internal surface to which the targeting nucleic acids are attached can have high heat conductivity to allow for the transfer of body heat to the reaction chamber. This surface can be placed so that it is in direct contact with the body. In some embodiments, there may be thermally conductive gap-filler or adhesives between the first external surface and the reaction chamber.

In some embodiments, the second external surface is on the opposite side to the first external surface. In some embodiments, the second external surface comprises an insulating material. The insulating material can minimize the heat loss to the surrounding environment. In some embodiments, thermal insulation of the chamber may be achieved either inside the device between the reaction chamber and the second external surface, and/or by the second external surface being composed of a thermally insulating material. Internally, the insulator may be an air gap, or a space filling material such as corrugated paper, or heat insulating foams. The second external surface itself may be composed of a material of low thermal conductivity, such as polypropylene or polyethylene.

In some embodiments, at least one external surface is transparent to provide a view of the reaction progress. In some embodiments, all external surfaces are transparent.

In some embodiments, the device further comprises a strap or a tape to fix the device to a human body.

In some embodiments, the targeting nucleic acids are immobilized to the inner surface of the device using methods and/or in manners described herein, for example, using methods and/or in manners described above for immobilizing targeting nucleic acids to a solid surface.

In some embodiments, the device further comprises a reaction mix in the reaction chamber. In some embodiments, the reaction mix comprises dNTPs, buffer, and at least one enzyme for isothermal amplification. In some embodiments, the isothermal amplification is TMA, NASBA, LAMP, HIP, HDA, RPA, SDA, or rolling circle amplification.

In some embodiments, the device is used for detecting presence of one or more target polynucleotides in a sample. In some embodiments, the device is used for quantifying the amount of one or more target polynucleotides in a sample.

In some embodiments, the device is used at a body temperature.

In some embodiments, one or more target polynucleotides are detected using the naked eye or a cell phone camera.

In some embodiments, the sample comprises a reference sequence and targeting nucleic acids for the reference sequence are immobilized to the inner surface.

In some embodiments, the targeting nucleic acids are positioned in a specific pattern according to a test serial number.

In some embodiments, in order to improve shelf-life length of the device, enzyme(s) required for amplification are stored as dried enzyme(s) until the actual execution of the test. In some embodiments, at the test initiation, two time-consuming processes take place in the device: (1) the reaction buffer is dispensed and mixed with the dried enzyme(s) to create a reagent mix (also referred to as “reaction mix”) that is sufficiently uniform; (2) the lysed sample is dispensed into the reaction chamber and incubated with the targeting nucleic acids (e.g., primer) clusters in the reaction chamber for a period of time such that there is a sufficient chance for the signal (i.e., one or more target polynucleotides) in the sample to bind to a matching cluster. By deferring the mixing of the enzymes with their buffer, the shelf life of the device can be extended, and storage does not require cooling.

After completion of the above two processes, the reaction mix is then dispensed into the reaction chamber to wash (e.g., 1, 2, 3, 4, 5, 6, 7, 8, 9, or 10 times or more) the reaction chamber, washing away unmatched polynucleotides and other unwanted materials (such as RNAses and DNAses that may be part of the sample, RNAse/DNAse blockers that may be part of the lysis buffer, and other reagents that may be required/useful for the lysis or binding but may interfere with the amplification) from the lysed sample, before amplification starts in the reaction chamber.

In some embodiments, in order to create a sufficient delay for these two processes, in a way which does not require the end-user to explicitly perform another operation, the device can include a membrane separating the reagent mix from the reaction chamber with a delay mechanism, i.e., a timed membrane. In some embodiments, after the concomitant induction of dispensing the lysed sample to the reaction chamber and mixing the reaction buffer with the dried enzyme(s), the timed membrane separating the reaction mix from the reaction chamber count-downs time, and after a certain delay, the timed membrane ceases to be a membrane and allows the reaction mix wash the lysed sample in the reaction chamber. In some embodiments, the timed membrane is made of a material that can be dissolved in the reaction buffer.

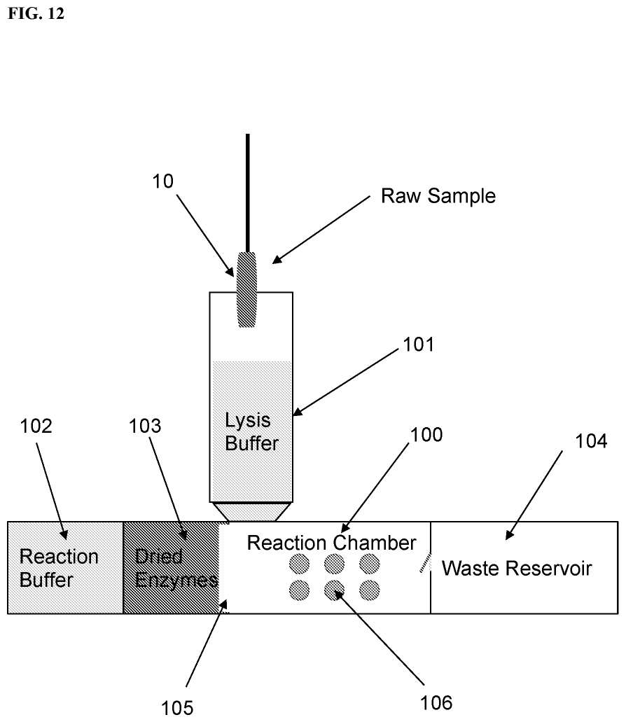

As illustrated in FIG. 12, a time membrane 105 can be used to separate the chamber that contains dried enzymes 103 from the reaction chamber 100. In the exemplary embodiment shown herein, the device includes: (1) a reaction chamber with primer clusters 106 immobilized (e.g., uniformly distributed) at the internal surface of the reaction chamber 100; (2) a sample container 101 containing lysis buffer and configured as a cylindroconical tube that has a proximal end attached to the reaction chamber 100, and an open distal end configured to receive a raw sample; (3) two adjacent reagent chambers, one containing reaction buffer 102 and the other containing dried enzymes 103 and separated from the reaction chamber 100 with a timed membrane 105; and (4) a waste reservoir 104 attached to the reaction chamber 100 downstream of the timed membrane 105 and the proximal end of the sample container 101 (e.g., at the opposite end of the reaction chamber 100 with respect to the timed membrane 105).

In some embodiments, as illustrated in FIG. 12, the reagent chamber that contains dried enzymes 103 is disposed downstream of the reagent chamber that contains reaction buffer 102, but upstream of the reaction chamber 100. The timed membrane 105 can be disposed upstream of the lysed sample entry point. As shown in FIG. 12, the two adjacent reagent chambers 102 and 103, the reaction chamber 100, and the waste reservoir 104 can be horizontally aligned, and the sample container 101 can be positioned on top of the reaction chamber 100 at an angle (e.g., about 90 degree) with respect to the reaction chamber 100.

In some embodiments, the flow from the sample container 101 to the reaction chamber 100 can be based on capillary action, gravity, and also be caused by a pressure generated by closing the sample container cap, e.g., either by turning a screw based cap or by pushing down a friction based cap.

In some embodiments, the flow from the reagent chamber 103 to the reaction chamber 100 is not gravity based. In some embodiments, the flow from the reagent chamber 103 to the reaction chamber 100 is not capillary action based. In some embodiments, the flow from the reagent chamber 103 to the reaction chamber 100 can be caused by a positive pressure in the reagent chamber (e.g., the reagent chamber 102 and/or 103 being held in an elastic container that has a higher pressure than the reaction chamber 100 and the waste reservoir 104. In some embodiments, the flow from the reagent chamber 103 to the reaction chamber 100 can be caused by a “negative pressure” in the waste reservoir 104. For example, the waste reservoir can apply some suction power, such as using a hygroscopic materials

Although the sample container 101 is configured as a cylindroconical tube in FIG. 12, it can be in other shapes that have a height. In some embodiments, the sample container 101 is configured to receive a raw sample carried by a carrier 10 (e.g., a swab), as shown in FIG. 12.

In some embodiments, the sample container 101 can be manufactured, packed, shipped, and/or stored as a separate item from the rest of the device, and then attached onto the reaction chamber 100 prior to performing a test with the device.

In some embodiments, the device does not include a sample container 101, and a processed sample (e.g., a lysed and/or purified sample) is dispensed into the reaction chamber 100 directly. In some embodiments, the device does not include reagent compartments 102 and 103, and the reaction mix is dispensed into the reaction chamber 100 directly, or the reaction chamber 100 comprises a pre-filled reaction mix.

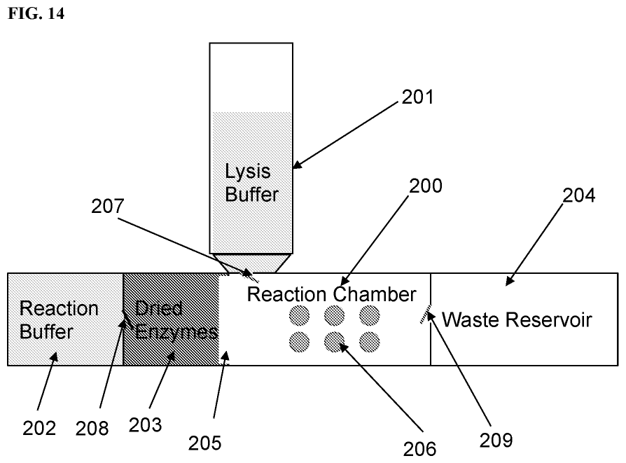

In some embodiments, the device can include three gates, as shown in FIG. 14. In the exemplary embodiment shown in FIG. 14, the first gate 207 is located at the proximal end of the sample container 201 which controls fluid flow from the sample container 201 to the reaction chamber 200; the second gate 208 is located on the wall that separates the two adjacent reagent chambers 202 and 203; and the third gate 209 is located on the wall that separates the reaction chamber 200 and the waste reservoir 204. In some embodiments, after the two gates shown in FIG. 14 (i.e., the first gate 207 that allows lysed sample to flow into the reaction chamber, and the second gate 208 that allows the reaction buffer to mix with the dried enzymes) are opened, the timed membrane 205 starts dissolving, thereby creating a delay that can be calibrated by the width of the membrane. In some embodiments, instead of a dissolving membrane, different timed gates can be used (e.g., a mechanical gate) which can be timed and controlled using different methods (e.g., pressure building, springs, electronic timer, Bluetooth signal sent from the phone app, etc.).

In some embodiments, one or more gates described herein pivot open, or slide open (such that the one or more gates can be received within adjacent wall). In some embodiments, one or more gates described herein are frangible portions that just break once reaching threshold.

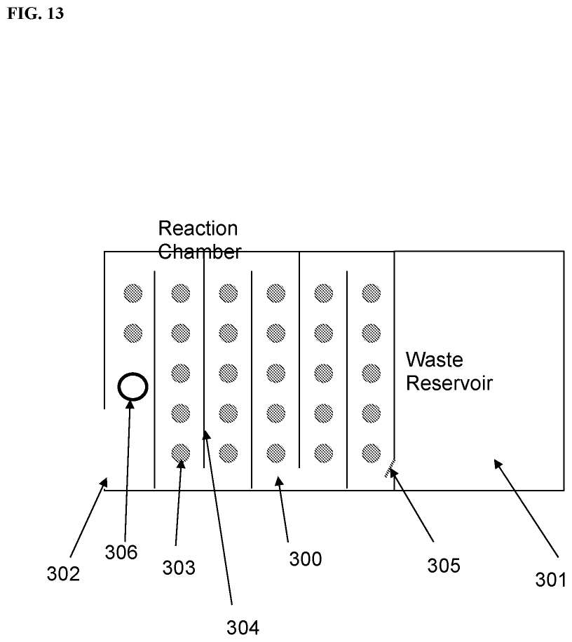

In some embodiments, instead of having a single vacuous volume, the reaction chamber 300 can include a plurality of internal baffles 304 defining a nonlinear (e.g., twisting) flow path from the timed membrane 302 and/or the lysed sample entry point 306 to the waste reservoir 301, forcing the lysed sample to traverse each of the primer clusters 303 in the reaction chamber 300, as exemplified in FIG. 13 in a top view. Different ratios and sizes of the path vs cluster sizes can achieve different trade-offs between optimal sensitivity (i.e., providing the best chance for each signal polynucleotide to be in close proximity to its associated primer cluster) and other design objectives (e.g., ease of manufacturing, efficiency of flow in the reaction chamber, etc.).

In some embodiments, as illustrated in FIG. 14, the single action of detaching the sample container 201 (e.g., the cylindroconical tube with the lysed sample) from the reaction chamber 200 prior to use of the device can have two effects: (1) open the gate 207 between the very same sample container 201 and the reaction chamber 200, allowing the lysed sample to flow into the reaction chamber 200 just before the sample container 201 is detached, and (2) open the gate 208 between the reagent chamber 203 that contains dried enzyme(s) and the reagent chamber 202 that contains reaction buffer.

In some embodiments, the device is configured to achieve the following one or more of the following objectives: (1) allow enough lysed sample to enter the reaction buffer; (2) do not overflow the waste reservoir and leave enough space for the reagent mix to wash the reaction chamber several times; and (3) do not allow the sample container drip after detaching it from the rest of the device.

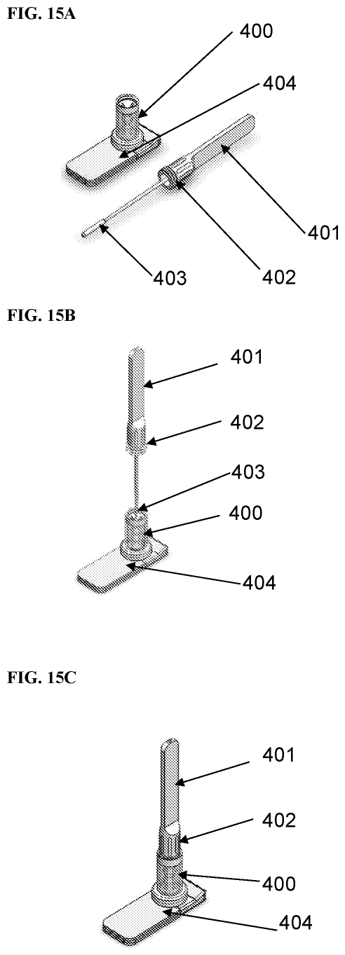

In some embodiments, the device further includes a screw cap 402 which can be screwed onto the open distal end of the sample container 400 to seal it. In some embodiments, a swab handle 401 is attached to the screw cap 402, as illustrated in FIG. 15A. As shown in FIGS. 15B and 15C, after placing the swab 403 in the sample container 400 (e.g., the sample tube), an end-user can proceed to screw the screw cap 402 on the sample container 400 (e.g., the sample tube), thereby creating some pressure inside the sample container 400 (e.g., the sample tube). After the screw cap 402 is fully screwed, it reaches a stop and can no longer rotate over the sample container 400's head, so that the continued motion of rotating the swab handle 401, in the same direction, now rotates the sample container 400's proximal end over the reaction chamber 404, thereby first opening the two gates illustrated in FIG. 14 (i.e., the first gate 207 that allows lysed sample to flow into the reaction chamber, and the second gate 208 that allows the reaction buffer to mix with the dried enzymes), and then detaching the sample container 400 (e.g., the sample tube). As soon as the gate between the sample container 400 (e.g., the sample tube) and the reaction chamber 404 is opened, the pressure generated in the sample container 400 (e.g., the sample tube), together with the capillary pull of the reaction chamber 404, transfers the lysed sample into the reaction chamber 404, equalizing again the sample container 400's pressure with the ambient pressure. In some embodiments, once the sample container 400 (e.g., the sample tube) is detached, the sample container 400 is completely closed at its distal end and having only a small hole at its proximal end with no additional liquid drips.

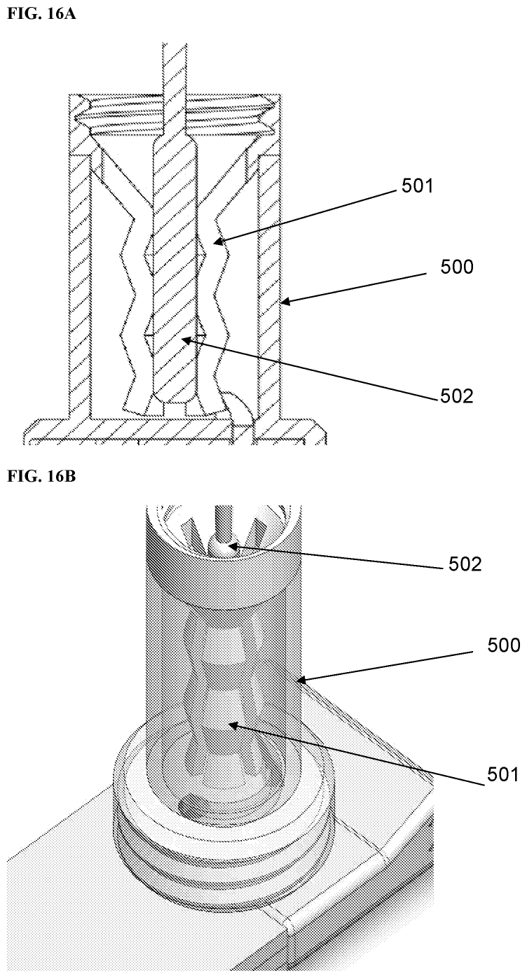

In some embodiments, the sample container 500 can include a plurality of undulating interior sidewalls 501 with peaks and valleys defining an internal channel, as illustrated in FIGS. 16A and 16B. In the exemplary embodiments shown herein, each peak of the sidewalls 501 has the same dimensions, and defines the narrowest point of the sample container 500 (e.g., the sample tube). When the swab 502 is pushed into the internal channel defined by the undulating interior sidewalls 501 of which all of the peaks are already fully immersed in the lysis buffer, the raw sample can be squeezed at each peak (narrowest point) of the interior sidewalls 501, where after each squeezing, the swab 502 gets to reabsorb lysis buffer, only to be squeezed again at the next peak (narrowest point).

Usually, in suspended NAAT tests, there's a delicate process of buffer exchange, where one wishes to replace the buffer to get rid of the lysis buffer together with all of the undesired elements of the raw sample and introduce the enzymes while keeping the polynucleotide signal as much as possible. For the devices described herein, the washing procedure is straightforward, as the desired signal is already bound to the internal surface of the reaction chamber. The devices described herein also allow getting rid of non-signal polynucleotides (i.e., non-targeting polynucleotides) while concentrating the signal (i.e., the targeting polynucleotides) found in the lysed sample.

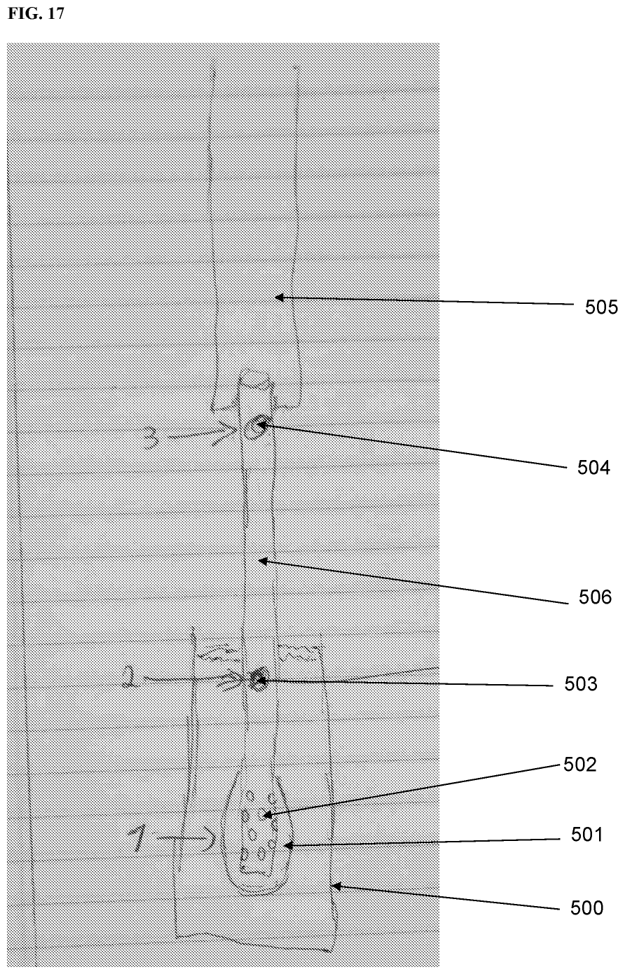

In some embodiments, the swab is configured to generate a flow of the lysis buffer through the swab's proximal end in order to move any raw sample caught in the swab's tip to the lysis buffer suspension in the sample container 500, as shown in FIG. 17. In the exemplary embodiment of the swab shown in FIG. 17, the swab's rod 506 can be designed as a tube with the following holes: (1) at the tip (i.e., the proximal end) of the swab, under the cotton-like fibers 501, the tube has many tight holes 502 and functions similar to a strainer; (2) just above the wad of fibers 501, the swab has a lysis buffer inlet 503 with one directional valve that can allow the entry of lysis buffer into the tube; (3) at the other end (i.e., the distal end) of the swab, just before the plunger 505, there can be an air hole 504 that can allow air to escape when the lysis buffer enters into the tube via the lysis buffer inlet 503. The air hole 504 can be covered and sealed immediately when the plunger 505 is pushed down.

### Methods

In certain aspects, provided herein are methods of quantifying and/or detecting one or more target nucleic acids using a device provided herein.

In some aspects, the present disclosure provides a method of quantifying one or more target polynucleotides in a sample, the method comprising: (a) providing a device described herein; (b) dispensing a sample to the reaction chamber; (c) incubating the device at a temperature for a period of time such that the presence of a target polynucleotide in the sample results in the generation of a cluster of immobilized amplicons on the internal surface of the reaction chamber at the predetermined location at which the targeting nucleic acid specific for that target polynucleotide was immobilized; and (d) counting the number of distinct clusters of amplicons for each target polynucleotide at the pre-determined location to quantify the amount of each target polynucleotide in the sample.

In some aspects, the present disclosure provides a method of detecting the presence of one or more target polynucleotides in a sample (if any are present), the method comprising: (a) providing a device described herein; (b) dispensing a sample to the reaction chamber; (c) incubating the device at a temperature for a period of time such that the presence of a target polynucleotide in the sample results in the generation of a cluster of immobilized amplicons on the internal surface of the reaction chamber at the predetermined location at which the targeting nucleic acid specific for that target polynucleotide was immobilized; and (d) detecting the position of clusters of amplicons on the solid surface to detect the presence of one or more target polynucleotides in the sample.

In some embodiments, the methods described herein further comprise dispensing a reaction mix to the reaction chamber prior to the step (c).

In some embodiments, the methods described herein further comprise, prior to the step (b), obtaining a raw sample with the swab attached to the screw cap, screwing the screw cap on the distal open end of the sample container until it is fully screwed, and rotating the swab handle to detach the sample container which opens the gate controlling flow of the lysed sample into the reaction chamber and the gate controlling mixing of reaction buffer with dried enzymes.

In some embodiments, the device is incubated at an ambient temperature (e.g., about 37° C. to about 42° C.). In some embodiments, the device is incubated on a human body, such as being placed within the arm-pit space, even above a shirt or a light sweater etc. The devices described herein can reach the desired temperature of over about 36.0° C. after proper incubation on a human body.

In some embodiments, the methods described herein comprise a step of slowly streaming all of the lysed sample over the primer clusters. The rate should be slow enough for the targeting polynucleotides to have a reasonable chance to bind the primers immobilized to the internal surface of the reaction chamber. One advantage of such step is that it allows using all of the lysed sample, rather than just one full volume of the reaction chamber, and thus can significantly improve sensitivity. Sample polynucleotides that match a primer cluster hybridize to the primer cluster and are henceforth fixed to the internal surface of the reaction chamber. By streaming all of the lysed sample, the primer clusters immobilized to the internal surface of the reaction chamber get a chance to scan all of the lysed sample for a matching signal polynucleotide (i.e., a matching targeting polynucleotide), rather than just a single chamber-volume, thereby concentrating a lysed sample from a bigger volume.

In some embodiments, the devices and methods provided herein can be used for the massive multiplexing of Nucleic Acid Amplification Tests (NAATs), in the sense that there is no limitation of the number of unique primer pairs used in the same reaction compartment, except for the limitation of physical space on the surface. Unique targeting nucleic acids (e.g., pairs of primers) for a specific target polynucleotide are separated in space, positioned in predetermined coordinates, conjugated to the surface, in separate spots. The separation into defined and confined positions prevents cross talk between the pairs of primers, eliminates primer dimers, and precludes the undesired use of one primer as a template to the other, as well as the general forming of a “hairball” of primers that would interrupt the reaction. The usage of predetermined locations enables the use of a uniform label, since the identity of an amplified target is resolved through its specific location and not by the wavelength of a fluorophore (as in suspended qPCR where fluorescence channels form a limitation on the number of different tests one can perform together in the same volume).

In one embodiment, the devices and methods provided herein can be used for digital quantitative parallel oligonucleotide amplification (e.g., digital qPCR, digital RT-qPCR, digital TMA, NASBA, LAMP, HIP, HDA, RPA, SDA, or exponential/linear rolling circle amplification, TMSD-mediated linear/nonlinear HCR, etc.). Each molecule of the marker being quantified can seed its own cluster on the surface; hence the number of distinct clusters thus formed, correlates with the number of molecules bearing this marker in the tested sample. This form of digital quantitative oligonucleotide amplification is cheaper and has a smaller footprint compared to existing methods. Such devices and/or methods further allow high throughput, parallel, digital amplification as multiple markers can be handled in this way on a single surface.

In some embodiments, the devices and methods described herein can be used for the detection of any polynucleotide sequence. The method enables parallel detection and quantification of many species of target polynucleotide sequences in a single small reaction volume requiring a very small sample volume.

In some embodiments, the devices and methods described herein can be used for the detection and therefore for diagnostics of pathogen polynucleotide sequences. In some embodiments, the devices and methods described herein enable testing in one run many hypotheses (e.g., different types of viruses, bacteria, cell free DNA, etc.).

In some embodiments, the devices and methods described herein can be used for the detection of known SNPs, insertions, deletions, inversions, translocations, or trisomies (e.g., in cell free DNA) for diagnostics or monitoring in various applications (e.g., planned parenthood, cancer, etc.).

In some embodiments, the devices and methods described herein can be utilized for detection, quantification, and determination of the origin of cell free DNA for diagnostics and monitoring in different applications (e.g., cancer, neurodegenerative disease, liver disease, heart attack, etc.).

In some embodiments, the devices and methods described herein can be used on the bed side or at home for self-diagnosis or telemedicine.

In some aspects, a device provided herein can be used in the performance of a method of quantifying one or more target polynucleotides in a sample, the method comprising: (a) contacting a sample to a solid surface on which a plurality of targeting nucleic acids (e.g., primers) are immobilized, wherein targeting nucleic acids specific for different target polynucleotides are immobilized at different predetermined locations on the solid surface; (b) performing an amplification process on the solid surface such that the presence of a target polynucleotide in the sample results in the generation of a cluster of immobilized amplicons on the solid surface at the predetermined location at which the targeting nucleic acid specific for that target polynucleotide was immobilized; and (c) counting the number of distinct clusters of amplicons for each target polynucleotide at the pre-determined location to quantify the amount of each target polynucleotide in the sample.

In some aspects, a device provided herein can be used in the performance of a method of detecting the presence of one or more target polynucleotides in a sample (if any are present), the method comprising: (a) contacting a sample to a solid surface on which a plurality of targeting nucleic acids (e.g., primers) are immobilized, wherein targeting nucleic acids specific for different target polynucleotides are immobilized at different predetermined locations on the solid surface; (b) performing an amplification process on the solid surface such that the presence of a target polynucleotide in the sample results in the generation of a cluster of immobilized amplicons on the solid surface at the predetermined location at which the targeting nucleic acid specific for that target polynucleotide was immobilized; and (c) detecting the position of clusters of amplicons on the solid surface to detect the presence of one or more target polynucleotides in the sample.

In some embodiments, the targeting nucleic acid comprises a primer. In some embodiments, only forward primers or reverse primers specific to the target polynucleotide are immobilized to the solid surface. In some embodiments, other primers that form a primer pair with the immobilized forward primers or reverse primers are contacted to the solid surface prior to step (b). In some embodiments, both forward and reverse primers specific to the one or more target polynucleotides are immobilized to the solid surface. In some embodiments, the primers comprise a set of nested primers for the same target polynucleotide. In some embodiments, one cluster of primers spreads into another cluster of primers within the set of nested primers for the target polynucleotides

In some embodiments, the targeting nucleic acids are uniformly immobilized to the predetermined location of the solid surface. In some embodiments, the targeting nucleic acids are immobilized to the solid surface as an array of targeting nucleic acid clusters, with each targeting nucleic acid cluster located at one of the predetermined locations on the solid surface. In some embodiments, the targeting nucleic acid clusters are spatially separated from each other on the solid surface. In some embodiments, multiple clusters of targeting nucleic acids for the same target polynucleotides are immobilized on the solid surface. In some embodiments, multiple clusters of targeting nucleic acids for detecting more than one target polynucleotides from the same cell, organism, or pathogen are immobilized to the solid surface. In some embodiments, each cluster of targeting nucleic acids comprises at least 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 104, 105, 106, 107, 108, 109, 1010, 1011, 1012, 1013, 1014, 1015, 1016, 1017, 1018, 1019, or more copies of the same targeting nucleic acid or set of targeting nucleic acids specific for the same target polynucleotide. In some embodiments, the targeting nucleic acid cluster is immobilized to a surface area with a size at least 0.01 mm², 0.0198 mm², 0.02 mm², 0.03 mm², 0.04 mm², 0.05 mm², 0.06 mm², 0.07 mm², 0.08 mm², 0.09 mm², 0.1 mm², 0.2 mm², 0.3 mm², 0.4 mm², 0.5 mm², 0.6 mm², 0.7 mm², 0.8 mm², 0.9 mm², 1 mm², 2 mm², 3 mm², 4 mm², 5 mm², 6 mm², 7 mm², 8 mm², 9 mm², or 10 mm². In some embodiments, the cluster of targeting nucleic acid is in a square shape with a side of 141 micron.

In some embodiments, the targeting nucleic acids (e.g., primers) are immobilized to the solid surface via its 5′ end. In some embodiments, the targeting nucleic acids are covalently linked to the solid surface. In some embodiments, the targeting nucleic acids are 5′ DBCO modified and the solid surface is azide functionalized surface. In some embodiments, the targeting nucleic acids are 5′ amine modified and the solid surface is NHS functionalized surface. In some embodiments, the targeting nucleic acids are non-covalently linked to the solid surface. In some embodiments, the targeting nucleic acids are immobilized to the solid surface via passive absorption, streptavidin-biotin interaction, or hybridization. In some embodiments, the 3′ end of the targeting nucleic acids are free to elongate. In some embodiments, the targeting nucleic acids are immobilized on a 2-dimensional surface. In some such embodiments, each cluster comprises targeting nucleic acids at a density of at least 9/micron², e.g., at least 10/micron², 15/micron², 20/micron², 25/micron², 30/micron², 35/micron², 40/micron², 45/micron², 50/micron², 55/micron², 60/micron², 65/micron², 70/micron², 75/micron², 80/micron², 85/micron², 90/micron², 95/micron², 10²/micron², 5×10²/micron², 10³/micron², 5×10³/micron², 10⁴/micron², 5×10⁴/micron², or 10⁵/micron². In some embodiments, the targeting nucleic acids are immobilized on a 3-dimensional surface. In some such embodiments, each cluster comprises targeting nucleic acids at a density of at least 9/micron³, e.g., at least 10/micron³, 15/micron³, 20/micron³, 25/micron³, 30/micron³, 35/micron³, 40/micron³, 45/micron³, 50/micron³, 55/micron³, 60/micron³, 65/micron³, 70/micron³, 75/micron³, 80/micron³, 85/micron³, 90/micron³, 95/micron³, 10²/micron³, 5×10²/micron³, 10³/micron³, 5×10³/micron³, 10⁴/micron³, or 5×10⁴/micron³, 10⁵/micron³, 5×10⁵/micron³, 10⁶/micron³, or 5×10⁶/micron³. In some embodiments, the targeting nucleic acids are invading primers. In some embodiments, the invading primers are LNA, PNA, PTO, ZNA, invader probe, or INA.

In some embodiments, the one or more target polynucleotides are DNA. In some embodiments, the one or more target polynucleotides are a single-stranded polynucleotides. In some embodiments, the one or more target polynucleotides are double-strand polynucleotides. In some embodiments, the method further comprises denaturing any polynucleotides in the sample prior to step (b) to generate single-strand polynucleotides. In some embodiments, a bisulfite conversion of the DNA sample occurred before step (a). In some embodiments, the target polynucleotides are RNA. In some embodiments, reverse transcriptase is contacted to the sample before step (b). In some embodiments, the target polynucleotides are linked to a barcode at their 5′ end, 3′ end, or at both ends. In some embodiments, the methods described herein further comprise adding a barcode to the 5′ end, 3′ end, or both ends of the target polynucleotides in the sample before step (a).

In some embodiments, a nucleic acid purification step is performed on the sample prior to step (a). In some embodiments, no nucleic acid purification step is performed on the sample prior to step (a). In some embodiments, the step (a) comprises (i) annealing the targeting nucleic acid to the corresponding target polynucleotide in the sample, (ii) washing the solid surface to remove contaminates from the sample, (iii) contacting the solid surface with a reaction mix.

In some embodiments, the sample comprises a reference sequence, and targeting nucleic acids for the reference sequence are immobilized to the solid surface. In some embodiments, the sample is diluted prior to the step (a). In some embodiments, a pool of samples is used in the step (a).

In some embodiments, the amplification process is a semi-solid phase amplification with one end on the solid surface and the other end in suspension. In some embodiments, the amplification process is a solid phase amplification. In some embodiments, the solid phase amplification process is a bridge amplification.

In some embodiments, the amplification process is a stepwise thermal amplification. In some embodiments, the amplification process is a stepwise chemical amplification. In some embodiments, the amplification process is PCR or RT-PCR. In some embodiments, the amplification process is an isothermal amplification. In some embodiments, the isothermal amplification process is TMA, NASBA, LAMP, HIP, HDA, RPA, SDA, or rolling circle amplification. In some embodiments, the step (b) occurs at an ambient temperature. In some embodiments, the step (b) occurs at about 37° C. to about 42° C. In some embodiments, the step (b) occurs at human body temperature. In some embodiments, the step (b) occurs without a mechanical heating device. In some embodiments, heat induced by chemical exothermic reaction at the step (b). In some embodiments, the amplification is non-enzymatic amplification process. In some embodiments, the non-enzymatic amplification process is TMSD-mediated HCR.

In some embodiments, the clusters of amplicons are detected using naked eye, a phase microscope, IRIS, observable sediment formation, SPR, or electric conductivity.

In some embodiments, the methods described herein further comprises labeling the clusters of amplicons prior to the step (c). In some embodiments, the clusters of amplicons are labeled using a DNA binding dye. In some embodiments, the DNA binding dye is an intercalating dye or a groove binding dye. In some embodiments, the DNA binding dye is SYTO-9, SYTO-13, SYTO-82, SYBR Green I, SYBR Gold, EvaGreen, PicoGreen, Ethidium Bromide, Acridine, Propidium Iodide, Crystal Violet, DAPI, 7-AAD, Hoechst, YOYO-1, DiYO-1, TOTO-1, DiTO-1, Hydroxystyryl-Quinolizinium Photoacid, or styryl dye. In some embodiments, the clusters of amplicons are labeled by incorporating a labeled nucleotide. In some embodiments, the clusters of amplicons are labeled using a surface bound or suspended probe. In some embodiments, the surface bound probe is in the same cluster as the targeting nucleic acid. In some embodiments, the surface bound probe is bound by the 3′ end. In some embodiments, the probe is a taqman probe, molecular beacon, or scorpion. In some embodiments, the nucleotide or probe is labeled with biotin, DIG, dppz, Ruthenium(II) complex, gold, Crystal Violet, HRP+TMB, Alkaline phosphatase, palladium, platinum, magnetic nanoparticle, BSA-MnO2 NPs, graphene oxide, Carbon dots, or agents for electrochemical detection. In some embodiments, amplicons of adjacent clusters are labeled differently. In some embodiments, amplicons of different target polynucleotides are labeled the same but at predetermined locations.

In some embodiments, the labeled clusters of amplicons are detected by a scanner, a fluorescence microscope, or a camera, optionally wherein the camera is a cell phone camera. In some embodiments, the number of distinct clusters of amplicons for each target polynucleotide is counted during step (c) to quantify the amount of each target polynucleotide in the sample.

### Sample Preparation

In some embodiments, the one or more target polynucleotides are DNA. In some embodiments, the one or more target polynucleotides are a single-stranded polynucleotides. In some embodiments, the one or more target polynucleotides are double-strand polynucleotides. In some embodiments, methods described herein further comprises denaturing any polynucleotides in the sample prior to step (c) to generate single-strand polynucleotides. In some embodiments, a bisulfite conversion of the DNA sample occurred before step (b).

In some embodiments, the target polynucleotides are RNA. In some embodiments, reverse transcriptase is contacted to the sample before step (c).

In some embodiments, the sample is diluted prior to the step (b).

In some embodiments, the methods described herein further comprise a sample preparation step, for example, a nucleic acid purification step, a sample enrichment step, or a reverse transcription step for RNA target molecule. In some embodiments, the sample preparation step (e.g., sample lysis, enrichment, and/or purification, reverse transcription, etc.) can be done directly on the solid surface or within the devices described herein.

In some embodiments, the sample is a purified nucleic acid (e.g., a purified DNA sample, a purified RNA sample, etc.) sample. In some embodiments, the sample is an unpurified sample, and the methods described herein include a nucleic acid purification step prior to contacting the sample to a solid surface on which a plurality of targeting nucleic acids are immobilized. The sample can be purified using any known methods in the art for DNA or RNA purification, or using commercially available kits. In some embodiments, the sample is a raw sample, the sample can be lysed within the sample container of the devices described herein prior to dispensing into the reaction chamber and contacting with the internal surface of the reaction chamber on which a plurality of targeting nucleic acids are immobilized.

In some embodiments, the sample is an unpurified sample (e.g., a lysed sample using the devices described herein), and the surface-bound targeting nucleic acids (e.g., primers) are used to simplify sample purification. As the targeting nucleic acids are conjugated to a solid surface, they can be used as capturing probes in the sense that after incubation with a sample, the surface can be washed to remove all unbound material including unrelated oligos, nucleases, proteases, and other contaminations or potential reaction inhibitors, leaving behind mostly the target nucleic-acid molecules hybridized to the surface-immobilized targeting nucleic acids. After the wash, the reaction can go straight ahead on the solid surface. This process could be done repeatedly with more and more sample, thus enriching the target oligos on the surface. In case of RNA target, at this point a reverse transcriptase can be introduced to elongate the binding primers into the wanted cDNA.

In some embodiments, the step (b) comprises (i) annealing the targeting nucleic acid to the corresponding target polynucleotide in the sample, (ii) washing the solid surface to remove contaminates from the sample, and (iii) contacting the solid surface with a reaction mix.

In some embodiments, the devices and methods described herein can be used to detect and/or quantify one or more target polynucleotides in a pool of samples. For example, in some embodiments, the oligo sample can be extended with a specific oligonucleotide barcode, either from one end of the strand or from both ends. Following that, pooled samples can be applied to the solid surface (e.g., the internal surface of the devices described herein) on which a plurality of targeting nucleic acids (e.g., primers, or primers and probes) are immobilized. In some embodiments, the barcode is attached to one end of the sample oligo, and spots hold one primer to complement and elongate the barcode side and one primer to complement the target oligo (to be detected) side. To increase specificity, spots can hold probes specific to the target. In some embodiments, the barcode is attached to both ends of the sample oligo, and spots hold primers complementing the barcodes fitting to amplify the stretch held between them, and probes specific to the target. In some embodiments, for each sample, some spots are designed to amplify the stretch between the 5′ end barcode and a 3′ end target marker, some designed to amplify the stretch between a 5′ end target marker and a 3′ end barcode and some to amplify the stretch between the 2 barcodes. Probes specific for the target can be added to all types of spots. In some embodiments, the barcode attachment is done while an initial signal amplification is done.

### Immobilized Targeting Nucleic Acid Clusters

In some embodiments, the immobilized targeting nucleic acid clusters comprise immobilized primers. In some embodiments, only forward primers or reverse primers specific to the target polynucleotide are immobilized to a solid surface (e.g., the internal surface of the devices described herein). In some embodiments, when only one primer from a pair is immobilized to the solid surface (e.g., the internal surface of the devices described herein), the other primer that forms a primer pair with the immobilized forward or reverse primer is contacted to the solid surface prior to amplification step (c). In some embodiments, the other primer that forms a primer pair with the immobilized primer is generated at step (c) (e.g., in an SDA process). In some embodiments, both forward and reverse primers specific to the one or more target polynucleotides are immobilized to the solid surface (e.g., the internal surface of the devices described herein). In some embodiments, primers can be found both immobilized in separate clusters and in suspension. In some embodiments, suspension amplification uses general mode of amplification (e.g., using degenerate primers, or conserved/similar flanking regions) while solid-phase amplification is specific (e.g., using nested primers, specific primers, etc.). In some embodiments, both forward and reverse primers from a pair of primers that are specific for a target polynucleotide are generated at step (c).

In some embodiments, the primers are uniformly immobilized to the predetermined location of the solid surface (e.g., the internal surface of the devices described herein). In some embodiments, when primers are uniformly attached to a predetermined area, the amplification process is stopped before clusters merge into each other in order to count.

In some embodiments, the primers are immobilized to the solid surface as an array of primer clusters, with each primer cluster located at one of the predetermined locations on the solid surface (e.g., the internal surface of the devices described herein). In some embodiments, the primer clusters are spatially separated from each other on the solid surface (e.g., the internal surface of the devices described herein). Preferably, primers are conjugated densely enough to allow the intended amplicon growing from one primer, anneal to the other primer, and allowing the polymerization of its complement. In some embodiments, the targeting nucleic acids are immobilized on a 2-dimensional surface (e.g., the internal surface of the devices described herein). In some such embodiments, each cluster comprises targeting nucleic acids at a density of at least 9/micron², e.g., at least 10/micron², 15/micron², 20/micron², 25/micron², 30/micron², 35/micron², 40/micron², 45/micron², 50/micron², 55/micron², 60/micron², 65/micron², 70/micron², 75/micron², 80/micron², 85/micron², 90/micron², 95/micron², 10²/micron², 5×10²/micron², 10³/micron², 5×10³/micron², 104/micron², 5×10⁴/micron², 6×10⁴/micron², or 10⁵/micron². In some embodiments, the targeting nucleic acids are immobilized on a 3-dimensional surface. In some such embodiments, each cluster comprises targeting nucleic acids at a density of at least 9/micron³, e.g., at least 10/micron³, 15/micron³, 20/micron³, 25/micron³, 30/micron³, 35/micron³, 40/micron³, 45/micron³, 50/micron³, 55/micron³, 60/micron³, 65/micron³, 70/micron³, 75/micron³, 80/micron³, 85/micron³, 90/micron³, 95/micron³, 10²/micron³, 5×10²/micron³, 10³/micron³, 5×10³/micron³, 10⁴/micron³, or 5×10⁴/micron³, 10⁵/micron³, 5×10⁵/micron³, 10⁶/micron³, 5×10⁶/micron³, or 8×10⁶/micron³.

A spotted array is preferable to the uniformly covered surface in order to keep clusters confined to a defined area and avoid the merge of clusters, especially for continuous amplification. A spotted and aligned array can be preferable to allow for a more straightforward data analysis as clusters'pre-determined position and the total number of clusters is known. It can simplify data-analysis to decide whether a position is populated or not, and what is the fraction of populated positions.

In some embodiments, at least about 1, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 10⁴, 10⁵, 10⁶, 10⁷, 10⁸, 10⁹ or 10¹⁰ distinct primers are immobilized onto the surface (e.g., the internal surface of the devices described herein). In some embodiments, each primer cluster comprises at least about 11, 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, 2000, 2500, 3000, 3500, 4000, 4500, 5000, 5500, 6000, 6500, 7000, 7500, 8000, 8500, 9000, 9500, 104, 105, 106, 107, 108, 109, 1010, 1011, 1012, 1013, 1014, 1015, 1016, 1017, 1018, 1019, or more identical primer molecules.

In some embodiments, different clusters can hold: (1) same detection agent (e.g., primers) for robustness or for target counting (signal from more clusters indicates more targets found); (2) different detection agent for the same target for increased robustness and positive predictive value; or (3) different detection agents for different targets for parallel detections of different targets.

In some embodiments, multiple different targeting nucleic acids for detecting the same target polynucleotides are immobilized on the solid surface (e.g., the internal surface of the devices described herein). In some embodiments, multiple different targeting nucleic acids for detecting more than one target polynucleotides are immobilized on the solid surface (e.g., the internal surface of the devices described herein). These target polynucleotides can be from the same cell, organism, tissue, patient, or pathogen. In some embodiments. In some embodiments, the methods described herein further comprises a step of using logical “and” “or” gates of more than one target polynucleotides to increase specificity and sensitivity.

In some embodiments, the devices and methods described herein can be used to test multiple markers to increase sensitivity and specificity. In order to account for the low specificity that may occur (e.g., due to the low annealing temperatures, etc.), multiple markers can be used in combination as a logical “and” gate, or multiple clusters indicating the same marker is found multiple times. In some embodiments, to increase sensitivity, multiple markers may be used in combination as a logical “or” gate. In some embodiments, the primers comprise a set of nested primers for the same target polynucleotide. In some embodiments, one cluster of primers spreads into another cluster of primers within the set of nested primers for the target polynucleotides. In some embodiments, some adjacent clusters can hold nesting primers in such a way that only if the right target oligo is amplified, the amplification can continue to the next cluster. As in regular nesting PCR, this decreases false positive cases, because the target oligo can hold both sets of primers, whereas there is a low chance that a different oligo may hold both sets of primers.

In some embodiments, in order to enhance “and”/“or” specificity, clusters for the detection of multiple markers of the same pathogen can be used. Using logical “and” gates increases specificity, while using logical “or” gates improves sensitivity. Having multiple clusters, possibly each matching a distinct marker, for the same pathogen (detection target), allows balancing and enhancing both sensitivity and specificity, making the final decision in the application or in the cloud, e.g. by a generic threshold “at least three out of the seven clusters”, or even more specifically, by assigning a specific weight for each detected cluster “and”/“or” combination of detected clusters.

In some embodiments, during the amplification process, amplified signal is released to the suspension and reseeds a different cluster (e.g., when TMA, NASBA, or SDA is used for the amplification step), the amplified signal can be different from the amplification initiating target.

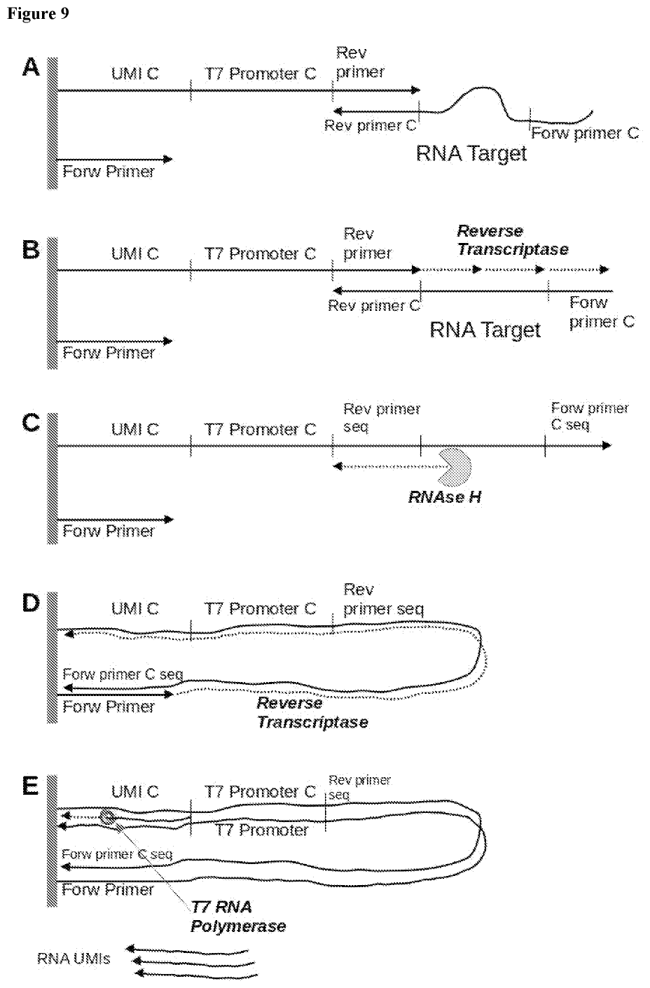

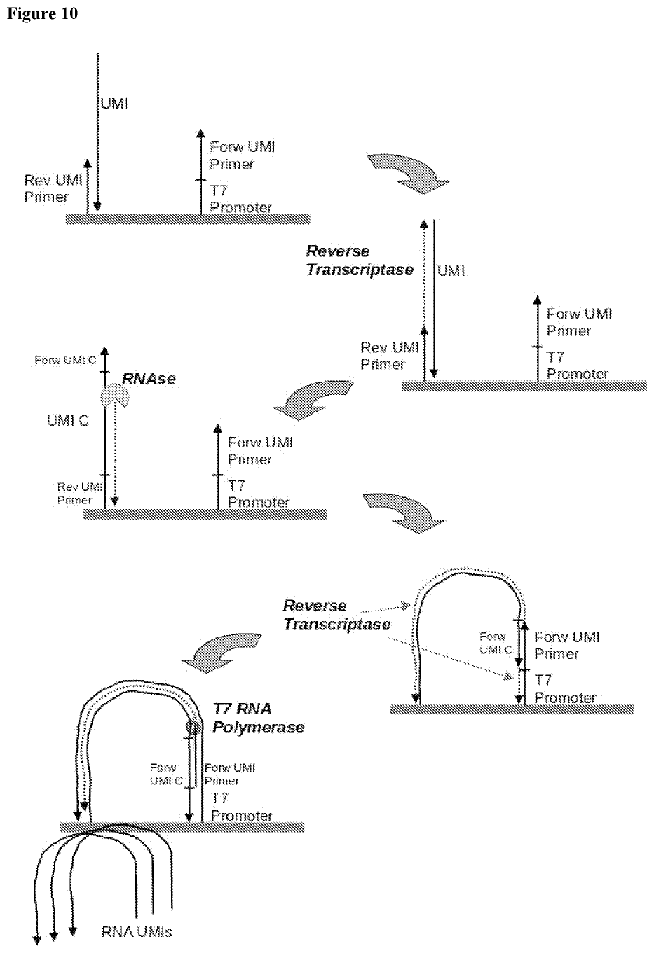

In some embodiments, a surrogate signal, different from the amplification initiating target, is amplified to generate the detectable signal. For example, in some embodiments, NASBA/TMA amplification method is used, and the signal RNA, as in a regular reaction, anneals to the reverse primer (FIG. 9A). The reverse primer is then elongated by the reverse transcriptase, according to the RNA target template (FIG. 9B). After the elongation, the RNA target, in the double strand RNA-DNA, is degraded by the RNAse H activity (FIG. 9C). The newly formed DNA reverse complement target anneals to the forward primer. The forward primer, in turn, elongates, and a dsDNA forms (FIG. 9D). One of the primers, in these embodiments, is designed to hold on its 5′ end a unique molecular identifier (UMI), and then a T7 promoter complementary stretch upstream to the primer. In these embodiments, the dsDNA holds the T7 promoter upstream to the UMI. Then the T7 polymerase generates copies of the UMI to be released to the reaction suspension (FIG. 9E). These UMI RNA copies find designated primer clusters for each specific UMI (FIG. 10). In some embodiments, SDA method is used for amplification, and the nick is designed to form on the target side. The primer is designed such that the UMI is at the 5′ tail. The target complementary to the primer is nicked and elongated according to the UMI template. In some embodiments, each UMI cluster type is generated of a single target molecule and as such, each UMI cluster formed can be count as one original target found in the sample. In some embodiments, each UMI cluster type is generated of at list a single target molecule and as such, each UMI cluster formed can be count as, at least, one original target found in the sample (e.g., 1 or more original targets). In some embodiments, the transition from original target to UMI is done in suspension.

In some embodiments, after the first target oligo seeds a UMI cluster, the option for a second target oligo to reseed the UMI type transition cluster is prevented. In some embodiments, the mechanism to achieve said prevention is by initiating local amplification (e.g., HIP), as to rapidly populate the cluster.

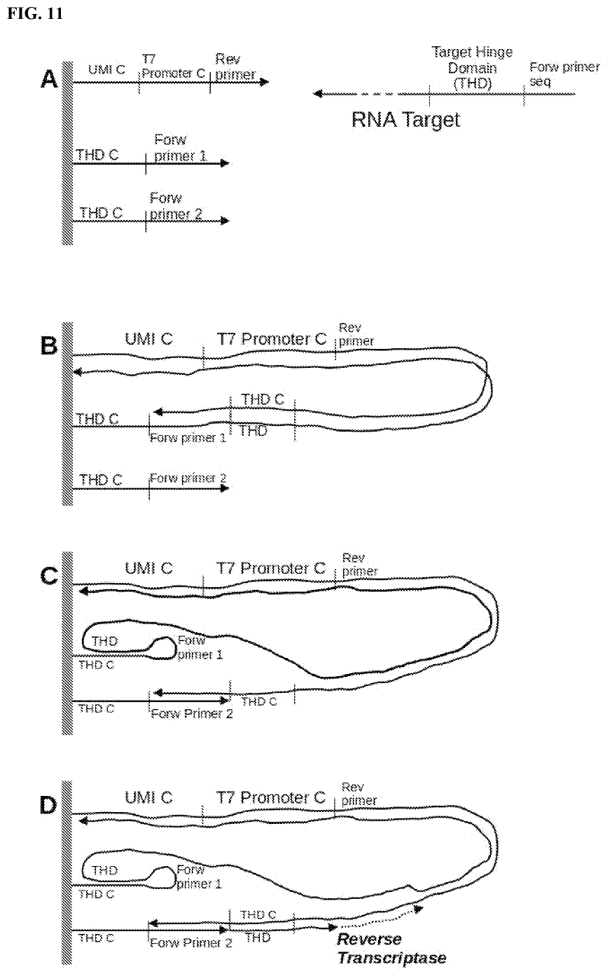

The forward primer has an additional domain at the 5′ end, which is complementary to a sequence of bases which are part of the RNA Target sequence, located upstream to the target's reverse primer complementary domain (that is, closer to the 5′ of the RNA target sequence), which is identified below as the Target Hinge Domain complementary (THD C) (FIG. 11A).

In some embodiments the THD C is an invading stretch (e.g. made in full or in part with PNA, LNA, etc.).

A dsDNA is formed as described above, i.e., reverse transcriptase elongates the reverse primer, RNAse H degrades the target RNA strand, the forward primer binds the 3′ end of the strand extending the reverse primer, and reverse transcriptase elongates the forward primer (FIG. 11B).

The complementary sequence thus constructed, extending forward primer 1, contains both a THD and a THD-complementary domain, separated by the forward primer sequence.

Additional forward primers are immobilized on the surface, and an energetically favorable configuration is for the THD-complementary domain of the forward primer 1 strand to pair with the THD on the same strand, while a distinct forward primer 2 molecule hybridizes with the forward primer complementary sequence on the 3′-end of the reverse complement target strand (FIG. 11C).

Reverse transcriptase can then elongate forward primer 2 against the reverse complement target strand, thereby separating the forward strand extended from forward primer 1 from the reverse complement strand. After elongation, the forward primer 2 strand has both a THD domain and a THD-complementary domain, so that the process can continue with yet another forward primer (FIG. 11D).

The forward primer 1 strand, which is separated from the reverse primer strand by the reverse transcriptase, can anneal to a different reverse primer immobilized on the surface.

Thus an exponential reaction takes place, eventually blocking all the reverse primers within the confines of the cluster, and preventing additional targets from hybridizing into a cluster whose UMI has already been triggered.

In some embodiments, a set of primer clusters are designed, each being the nested set of primers of the specific stretch of oligo screened for. In some embodiments, during the amplification process, amplified signal is not released to the suspension, and the nested primer cluster is concatenated in a way that one can spread in to the other.

In some embodiments, the sample comprises a reference sequence, and primers for the reference sequence are immobilized to the solid surface.

In some embodiments, the targeting nucleic acids are immobilized to the solid surface (e.g., the internal surface of the devices described herein) via its 5′ end. In some embodiments, the targeting nucleic acids are immobilized to the solid surface (e.g., the internal surface of the devices described herein) via either 5′ or 3′ end, and a non-enzymatic amplification process is used at the step (c). In some embodiments, the targeting nucleic acids are covalently linked to the solid surface (e.g., the internal surface of the devices described herein). In some embodiments, the user can change the analyzed target (i.e., customize the markers) between runs so that the devices and methods described herein can be used in a more flexible way. One exemplary method that can be employed to select and cover the surface with the desired targeting nucleic acids is the use of click chemistry-covered surfaces functionalized with azide, NHS, etc. Targeting nucleic acids are designed with the appropriate modification on the 5′ end (e.g., DBCO for azide functionalized surfaces, or amine for NHS functionalized surfaces, etc.). The first step in such an exemplary method is applying the 5′ modified targeting nucleic acids on the functionalized surface to allow for the conjugation of the targeting nucleic acids to the surface. The surface is then washed thoroughly to get rid of any unbound targeting nucleic acid, and all of the unused functional groups are blocked for any non-specific binding. Additional methods for covalently linking the targeting nucleic acids to the solid surface (e.g., the internal surface of the devices described herein) include but are not limited to, e.g., azide-alkyne; Amine-reactive-NH2+NHS ester/Imidoester/Pentafluorophenyl ester/Hydroxymethyl phosphine/Epoxide/Isocyanate; Carboxyl-to-amine reactive —COOH+Carbodiimide (e.g., EDC); Sulfhydryl-reactive—SH+Maleimide/Haloacetyl (bromo-, chloro-, or iodo-)/Pyridyl disulfide/Thiosulfonate/Vinyl sulfone; Aldehyde-reactive (e.g., oxidized sugars, carbonyls) —CHO+Hydrazide/Alkoxyamine/NHS ester; Hydroxyl (nonaqueous)-reactive —OH+Isocyanate; photoreactive cross linking( e.g., aryl azides+nucleophile (e.g., primary amine), diazirine+amino acid side chain or peptide backbone), etc. In some embodiments, the targeting nucleic acids are 5′ DBCO modified and the solid surface (e.g., the internal surface of the devices described herein) is azide functionalized surface. In some embodiments, the targeting nucleic acids are 5′ amine modified and the solid surface (e.g., the internal surface of the devices described herein) is NHS functionalized surface.

A different exemplary method for covering the surface (e.g., the internal surface of the devices described herein) with the user primers is elongating the already attached primers, populating the surface according to a template of the user design. This template can be designed such that its 3′ stretch complements the primers already immobilized to the solid surface and the 5′ stretch complements the intended primer. In this exemplary method, the first step is inserting a high concentration of both of the user-designed DNA strands, annealing to the already existing, surface-attached, primers, and elongating using DNA polymerase to generate the desired primers. All templates are then denatured and washed thoroughly off the reaction compartment.

In some embodiments, wherein steps of denaturation in aggressive heat or chemical reagent are not used, the targeting nucleic acids are non-covalently linked to the solid surface (e.g., the internal surface of the devices described herein), e.g., via passive absorption, streptavidin-biotin, hybridization, etc.

In some embodiments, the 3′end of the targeting nucleic acids are free to elongate.

In some embodiments, the targeting nucleic acids are invading primers, e.g., LNA, PNA, PTO, ZNA, invader probe, or INA.

In certain embodiments the surface can be any solid support. In some embodiments, the surface is the surface of a flow cell. In some embodiments, the surface is a slide, a chip (e.g., the surface of a gene chip), a microwell plate, a plate, a tube, or a fluidic channel. In some embodiments, the surface is a bead (e.g., a paramagnetic bead). Cartridges formats can be varied, for example, a well plate (e.g. 96 well plate) can be used to digitally test different samples or different markers in every well. A different example is the use of fluidics channels, which can be integrated into a machine that allows for washing steps.

In some embodiments, the surface is an inner surface of the devices described herein. In some embodiments, the surface is a 3D polymer.

### Amplification

In some embodiments, the amplification process is a semi-solid phase amplification with one end on the solid surface (e.g., the internal surface of the devices described herein) and the other end in suspension. In some embodiments, the amplification process is a solid phase amplification. In some embodiments, amplification can occur both in suspension and in solid phase at the same time.

In some embodiments, the solid phase amplification process is a bridge amplification. The amplification process can be a stepwise thermal or chemical amplification, e.g., PCR, RT-PCR, etc. In some embodiments, the amplification process is an isothermal amplification, including but not limited to, e.g., TMA, NASBA, LAMP, HIP, HDA, RPA, SDA, or exponential/linear rolling circle amplification, etc. Isothermal amplification methods, such as HIP, LAMP, HDA, RPA, SDA, NASBA, TMA, etc., are known in the art. For example, HIP is described in Fischbach et al. (2017) Scientific Reports, 7:7683|DOI:10.1038/s41598-017-08067-x; low temperature LAMP is described in Cai et al. (2018) Anal. Chem. 90:8290-8294; HDA is described in Vincent et al. (2004) EMBO Rep. 5:795-800; TMA is described in Brentano and Mcdonough (2000) Nonradioactive Analysis of Biomolecules 374-380; RPA is described in Piepenburg O et al. (2006) PLOS Biology 4(7): e204. ; SDA is described in Walker GT et al. (1992) Nucleic Acids Research 7:1691-1696; NASBA is described in Deiman B. et al. (2002) Molecular Biotechnology 20:163-179, each of which is incorporated by reference herein in its entirety. In some embodiments, the amplification is non-enzymatic amplification process, e.g., TMSD-mediated linear/nonlinear HCR, etc.

In some embodiments, the amplification process occurs at an ambient temperature. In some embodiments, the amplification process occurs at about 37° C. to about 42° C. In some embodiments, the amplification process occurs at human body temperature. In some embodiments, the amplification process occurs without a mechanical heating device. In some embodiments, heat induced by a chemical exothermic reaction for the amplification process. Different primer configurations may be used for different amplification methods. For example, in some embodiments, stepwise thermal or chemical PCR amplification method is used, both of the primers specific to the intended target would be densely attached to the surface. The surface can be either spotted with an array of primer spots or be uniformly covered with the primers. In some embodiments, a chemical PCR process is used, and the sample is applied in a single strand form (for example, by using the Illumina sample denaturation and dilution protocol). In some embodiments, a thermal PCR process is used, and the first step is denaturation of double stranded DNA sample to generate single strand form. Alternatively, invading primers (e.g., LNA, PNA, PTO, ZNA, Invader probes, INA, etc.) can be used.

In some embodiments, annealing of the target polynucleotides to the primers can then take place. Incubation temperature and time should be adjusted according to the sample complexity, the amplification method, and the primers Tₘ. These parameters can impact the sensitivity and the specificity of the reaction. In some embodiments, the polymerase elongates the primers attached to a target after the annealing step. The annealing and elongation steps can be considered as the seeding step. In some embodiments, denaturation and washing steps can be applied to ensure that only covalently bound material stays in the system and ends the seeding step. In some embodiments, the washing step is not applied and continuous seeding occurs. If the washing step is done, new amplification reagent (e.g., PCR reagent) should be introduced to the reaction compartment (e.g., the reaction chamber described herein). In some embodiments, rounds of bridge amplification (e.g., bPCR) can generate clusters of the amplified target. Each cluster is seeded by (i.e., originated out of) a single target molecule seed. The number of clusters correlates with the number of target molecules found in the sample applied to the surface. The number of rounds depends on the size of cluster needed for detection according to the sensitivity of the detection method used. In certain embodiments, probes are used in the detection method, and extra steps allowing for specific probe annealing take place.

In some embodiments, the starting material is RNA, and no denaturation takes place in sample preparation and the elongation step in the seeding stage is done using reverse transcriptase.

In some embodiments, RPA isothermal amplification is used. In this case, the denaturation step is not relevant, washing steps are not necessary, and amplification does not require a heating device as the reaction can occur in an ambient temperature. This allows implementing the amplification process in a simple device, such as the devices described herein.

In some embodiments, TMA or NASBA isothermal amplification is used. In this case, the starting material is single stranded DNA or RNA (dsDNA is also possible if invading primers are used), and a low temperature, for example, 37-42° C., can be used. In some embodiments, human body temperature should suffice, and no heating device is needed.

### Labeling and Detection

In some embodiments, the methods described herein comprise labeling the clusters of amplicons prior to the detection step. In some embodiments, the clusters of amplicons are labeled using DNA binding dyes such as intercalating dye, groove binding dyes, etc., including but not limited to, e.g., SYTO-9, SYTO-13, SYTO-82, SYBR Green I, SYBR Gold, EvaGreen, PicoGreen, Ethidium Bromide, Acridine, Propidium Iodide, Crystal Violet, DAPI, 7-AAD, Hoechst, YOYO-1, DiYO-1, TOTO-1, DiTO-1, Hydroxystyryl-Quinolizinium Photoacid, or styryl dye.

In some embodiments, the clusters of amplicons are labeled by incorporation of an intrinsically fluorescent or labeled nucleotide, e.g., an intrinsically fluorescent or labeled dUTP, dGTP, dCTP, dATP, etc.

In some embodiments, the clusters of amplicons are labeled by a surface bound or suspended probe, e.g., a Taqman probe, molecular beacon, or scorpion, etc. In some embodiments, the surface bound probe is in the same cluster as the primer. In some embodiments, the surface bound probe is bound by the 3′ end. In some embodiments, the amplified amplicons are released to the suspension, and the detecting probes can be in separate clusters.

The probes, labeled dNTPs, and groove binding agents can be labeled with many different molecules for either immediate or for secondary reporting. For example, they can be labeled with biotin, DIG, and with either fluorescent, luminescent (e.g., dppz, Ruthenium (II) complex, etc.), colorimetric (e.g., gold, Crystal Violet, HRP+TMB, Alkaline phosphatase, palladium, platinum, magnetic nanoparticle, BSA-MnO2 NPs, graphene oxide, Carbon dots), or electrochemical detection.

In some embodiments, the clusters of amplicons are not labeled, and the detection is based on the change in physical qualities occurring due to the amplification on a specific spot e.g., change in the optical properties (e.g. refraction, opacity, etc.) (can be detected using phase microscopy, IRIS, etc.), observable sediment formation, SPR, electric conductivity, etc.

In some embodiments, amplicons of different target polynucleotides are labeled differently. In some embodiments, adjacent clusters are labeled differently. In some embodiments, amplicons of different target polynucleotides are labeled the same but at pre-determined locations.

In some embodiments, primer clusters bound by a targeting polynucleotide can change their color during the test to generate a biological test result pattern similar to a QR code, which can then be scanned by a camera (e.g., a smartphone camera) much like a QR code. In some embodiments, there can also be a QR/QR-like code pre-printed on the device, providing information about the test: e.g., which kind of test it is (e.g., respiratory, urinary, etc.), a serial/batch-id, a pattern-id, and/or an expiration date.

In some embodiments, the two patterns (i.e., the biological test result pattern and the pre-printed QR/QR-like code) can be scanned together in a single camera operation. In some embodiments, the device can include visual fiducials, to facilitate orientation/registration of the image scans.

In some embodiments, the methods further comprise collecting the geographic locations of the end-users from those who agree to share their geographic locations (e.g., using the smartphone's GPS). This can improve the test results, because by aggregating epidemiological data, the methods described herein can adapt the thresholds for deciding whether a set of markers indicate a specific pathogen for the current situation in the vicinity of the person being tested (e.g., for a northern hemisphere individual testing in July to get a flu diagnosis, multiple flu associated clusters must be “ON”, while for an individual testing in the midst of an area in which flu is strongly spreading, the test is nearly superfluous).

### EXAMPLES

### Example 1—Signal Amplification Using Bridge PCR

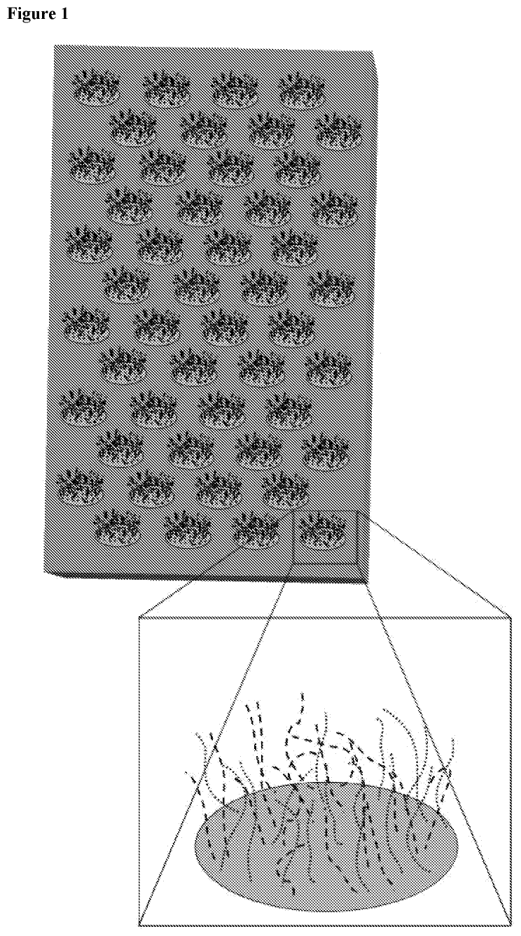

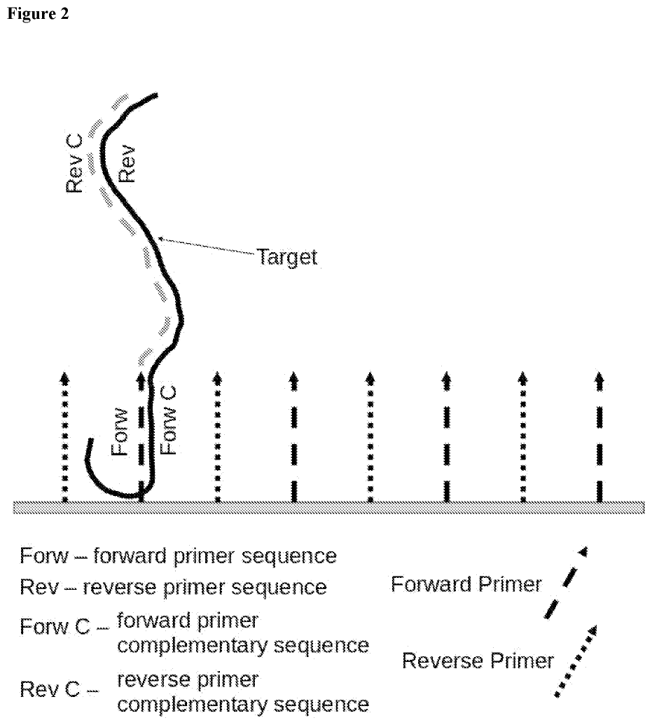

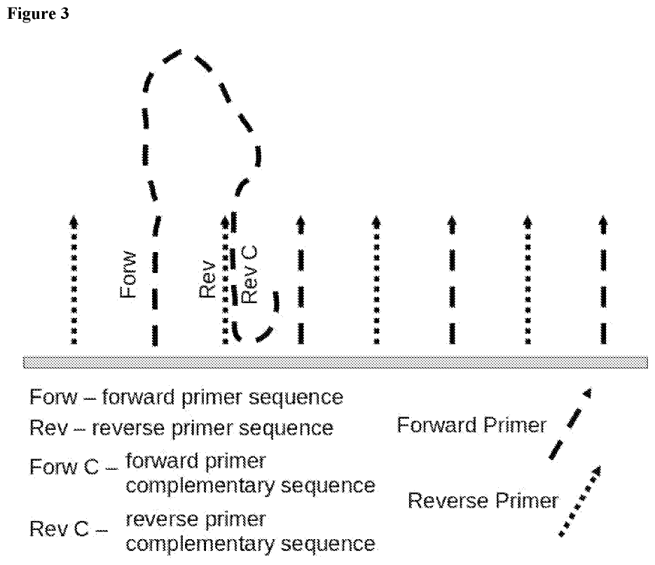

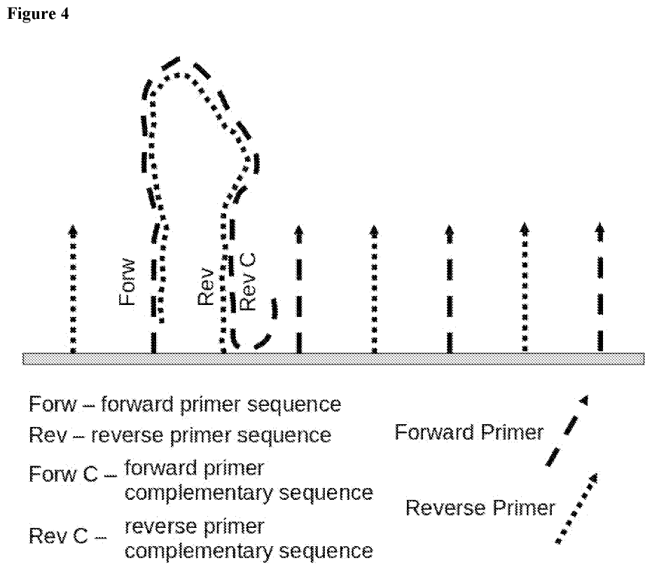

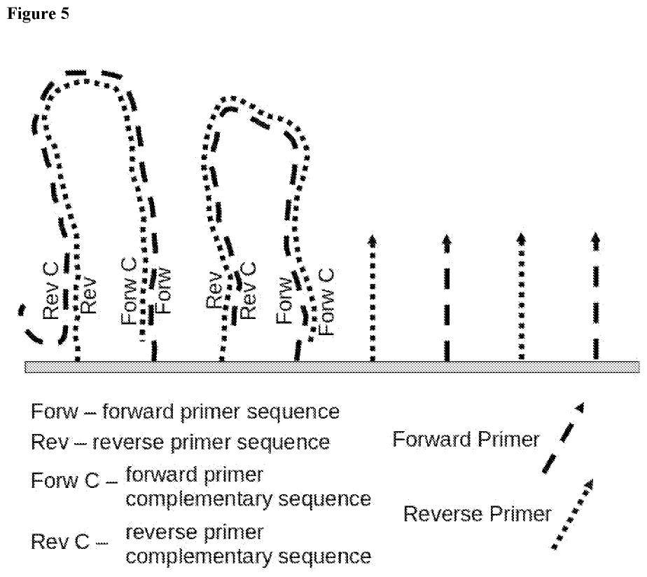

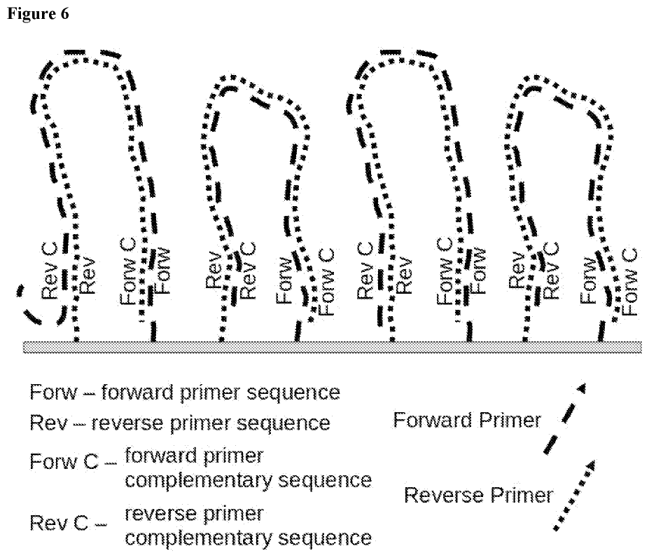

In some embodiments, the PCR process can be either an isothermal PCR or thermal steps-based PCR. In some embodiments, a set of spatially separated clusters, are positioned on a solid surface (FIG. 1). Each cluster can hold both the forward and reverse primers specific for one or more oligo targets. The dense primers in a cluster can create high local concentration of the specific primers. As seen in FIGS. 2 and 3, the target can find one of its corresponding primers and initiates a Polymerization reaction, generating the complement for the reverse primer found in the same cluster. As seen in FIGS. 4-6, after the displacement of the target the bridge PCR reaction is initiated, generating a cluster in the predefined location on the surface. At the end of the run, which of the clusters a PCR reaction occurred is detected,, and based on this information the targets are identified and quantified. In one embodiment, each square of surface holds many copies of two unique primers, specific for one molecular marker-oligo target. If an oligo target appears in the sample, a cluster appears on the appropriate square, and each cluster is an amplification of exactly one oligo strand. In some embodiments, each square surface holds an array of primer clusters that may be homogeneous, and specific to one oligo target.

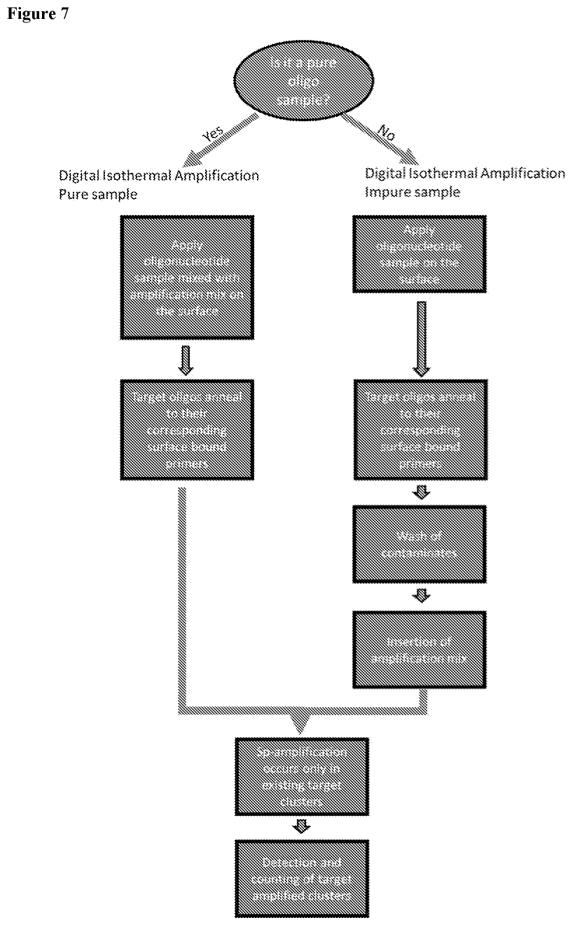

FIG. 7 shows an exemplary digital PCR (dPCR) flow chart.

### Example 2—TMA

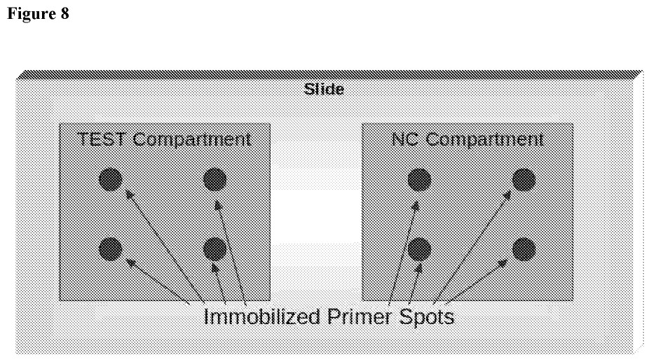

A 3D azide glass slide was used as the solid surface. 5′ DBCO modified reverse primers, were spotted in duplicates on the glass slide. The spotting pattern was repeated in 2 locations on the slide (FIG. 8). The glass with the spots was incubated overnight at RT (room-temperature) and then washed 3 times with 1.5×SSC, 0.1% SDS and 1 time with UPW.

The spotted glass was then blocked using 0.1% BSA and 0.2 mg/ml salmon sperm for 30 min at RT and then washed with UPW and dried. An NASBA mix was prepared using the amsbio NASBA wet kit according to the manufacturer protocol. Only forward primer was added to the mix. A labeled dUTP was added to the prepared mix. Before adding the enzyme cocktail to the NASBA mix, the mix was split to two. The enzyme cocktail was added to one and the other was supplemented with UPW instead to serve as the negative control. The full mix was applied on one of the spotting locations and the NC (negative control) mix was applied on the other spotting location. Both locations were then covered by cover slips and sealed using Photo glue. When the photo glue dried off, the slide was incubated in 41° C. for 90 min. After incubation, the glue and the coverslips were removed and the glass was washed 4 times using 1.5×SSC, 0.1% SDS. Then the glass was incubated for 15 minutes with two different probes—cy5 reverse primer probe and fam reverse complement target probe. The latter was composed of LNA bases in order to facilitate strand invasion. Probe mix was supplemented with 0.2 mg/ml salmon sperm to decrease nonspecific binding of the probes. After the incubation, washing was repeated and the glass was inspected using a fluorescence microscope. The microscope image of the full NASBA reaction demonstrated collocated fluorescence in the three channels that were imaged—Cy5, labeled dUTP, and FAM. The cy5 channel confirms that the reverse primers were successfully immobilized to the surface, and indicates the location of the spot. The collocated fluorescence in the labeled dUTP channel indicates that a polymerization of a nucleic acid strand was accomplished. The collocated fluorescence in the FAM channel confirms that the probe for the reverse target hybridizes with the said strand as expected. In contrast, the microscope image for the negative control spots, where the enzyme was missing, demonstrated fluorescence only in the cy5 channel, indicating that no polymerization took place in that compartment.

### Example 3—TMA

In a follow-up experiment, the two sets (TEST/NC) of spots held a mixture of both forward and reverse primers. No primers were in the solution mix. As described for the previous experiment, the mix was split in two: the full reaction (TEST) and no enzyme (NC). Imaging the three fluorescent channels in a microscope, the spot with both primers immobilized and with the NASBA enzyme demonstrated all three collocated channels, indicating successful amplification of the signal. The spot with both primers but without NASBA enzyme only showed a fluorescent channel in the cy5 channel, indicating the existence of the reverse primers, but no polymerization taking place.

### Example 4—PCR

In a similar fashion, spots of either reverse or forward primer were prepared. A PCR mix, with no primers, supplemented with labeled dUTPs, was prepared and applied on the conjugated primer spots. Single stranded DNA target was added in excess to the mix. The reaction was compartmentalized and sealed using BioRad frame seal for in situ PCR. The slide was loaded into a thermocycler using a “96 wells to slides” adapter for 1 round of polymerization. The frame seal was then removed and the slide washed, incubated with probes, washed again and results were inspected under a fluorescence microscope as described before for the NASBA reaction. Specific polymerization using 1 round PCR reaction on slide was done.

Primers were only in the spots covalently attached to the glass slide. Large excess of ssDNA target was used.. Inspection of the spot where the reverse primer was immobilized demonstrated a collocated signal for the three fluorescent channels, indicating successful polymerization of the reverse target in this spot. In contrast, when inspecting the spot in which the forward primer was immobilized, an irrelevant primer in these settings, no significant signal was visible, indicating no polymerization occurred.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A device comprising a first external surface, a second external surface that is different from the first external surface, an internal surface, a plurality of targeting nucleic acids immobilized to pre-determined locations on the internal surface, and a reaction chamber enclosed by the internal surface, wherein targeting nucleic acids specific for different target polynucleotides are immobilized at different predetermined locations on the internal surface.

2-3. (canceled)

4. The device of claim 1, wherein the first external surface comprises a material that is able to conduct heat, and the second external surface is on the opposite side to the first external surface and comprises an insulating material.

5-12. (canceled)

13. The device of claim 1, wherein (1) the targeting nucleic acids specific for each target polynucleotide are uniformly immobilized to the predetermined location of the internal surface; or (2) the plurality of targeting nucleic acids are immobilized as an array of targeting nucleic acid clusters, with each targeting nucleic acid cluster located at one of the predetermined locations on the internal surface.

14-27. (canceled)

28. The device of claim 1, wherein the device further comprises a reaction mix in the reaction chamber, and wherein the reaction mix comprises dNTPs, buffer, and at least one enzyme for isothermal amplification.

29-30. (canceled)

31. The device of claim 1, wherein the device further comprises a sample container, a reagent compartment, and a waste reservoir, each of which is connected to the reaction chamber.

32. The device of claim 31, wherein the device further comprises one or more valves selected from: (1) a first valve which controls fluid flow from the sample container to the reaction chamber; (2) a second valve which controls fluid flow from the reagent compartment to the reaction chamber,; and (3) a third valve which controls fluid flow from the reaction chamber to the waste reservoir.

33-34. (canceled)

35. The device of claim 31, wherein the sample container (1) contains lysis buffer; and/or (2) has a proximal end that is connected to the reaction chamber and an open distal end that is configured to receive a raw sample from a carrier.

36-38. (canceled)

39. The device of claim 31, wherein (1) the reagent compartment and/or the sample container is disposed upstream of the waste reservoir; (2) the reagent compartment is disposed upstream of the lysed sample entry point; (3) the reagent compartment, the reaction chamber, and the waste reservoir are horizontally aligned; and/or (4) the sample container is positioned on top of the reaction chamber at an angle with respect to the reaction chamber.

40-43. (canceled)

44. The device of claim 31, wherein the reagent compartment comprises a first reagent chamber and a second reagent chamber, and a fourth valve; and wherein the fourth valve controls fluid flow from the first reagent chamber to the second reagent chamber.

45. The device of claim wherein (1) the first reagent chamber contains reaction buffer, and the second reagent chamber contains dried enzymes, and wherein the reaction buffer dispenses into the second reagent chamber to dissolve the dried enzymes to form a reaction mix which then dispenses into the reaction chamber; (2) the second reagent chamber is disposed downstream of the first reagent chamber and upstream of the reaction chamber; (3) the first reagent chamber, the second reagent chamber, and the reaction chamber are horizontally aligned; and/or (4) the second valve controlling fluid flow from the reagent compartment to the reaction chamber controls the fluid flow from the second reagent chamber to the reaction chamber.

46-48. (canceled)

49. The device of claim 44, wherein the second valve controlling fluid flow from the reagent compartment to the reaction chamber is a timed valve, and wherein the timed valve is configured to open after a period of time after the first valve and the fourth valve are open.

50-55. (canceled)

56. The device of claim 31, wherein the reaction chamber (1) comprises a plurality of internal baffles defining a nonlinear flow path from the lysed sample entry point and/or the reaction mix entry point to the waste reservoir; and/or (2) has a height of 10-700 microns.

57. (canceled)

58. The device of claim 44, wherein the sample container (1) is configured to open the first valve when it is being detached, thereby allowing the lysed sample to flow into the reaction chamber before the sample container is detached; and/or (2) is configured to open the fourth valve when it is being detached.

59. (canceled)

60. The device of claim 44, wherein the device further comprises a screw cap that can be screwed to the distal end of the sample container to seal it.

61. The device of claim 60, wherein the screw cap is attached to the distal end of the swab on one side and to a swab handle on the opposite side such that the swab can be placed into the sample container with the proximal end immersed in the lysis buffer when the screw cap is screwed onto the sample container.

62. The device of claim 61, wherein the device is configured in a way that (1) continued motion of rotating the swab handle in the same direction after the screw cap is fully screwed opens the first valve and the fourth valve and then detaches the sample container; or (2) shoving the swab into the sample container opens the first valve and the fourth valve.

63. (canceled)

64. The device of claim 61, wherein the sample container comprises a plurality of undulating interior sidewalls with peaks and valleys, and wherein the interior sidewalls are configured in a way that the swab is squeezed at each peak of the interior sidewalls.

65-67. (canceled)

68. The device of claim 61, wherein the swab is configured to allow fluid flowing through the proximal end of the swab.

69-78. (canceled)

79. A method of quantifying one or more target polynucleotides in a sample, the method comprising: (a) providing a device of claim 1; (b) dispensing a sample to the reaction chamber; (c) incubating the device at a temperature for a period of time such that the presence of a target polynucleotide in the sample results in the generation of a cluster of immobilized amplicons on the internal surface of the reaction chamber at the predetermined location at which the targeting nucleic acid specific for that target polynucleotide was immobilized; and (d) counting the number of distinct clusters of amplicons for each target polynucleotide at the pre-determined location to quantify the amount of each target polynucleotide in the sample.

80. A method of detecting the presence of one or more target polynucleotides in a sample (if any are present), the method comprising: (a) providing a device of claim 1; (b) dispensing a sample to the reaction chamber; (c) incubating the device at a temperature for a period of time such that the presence of a target polynucleotide in the sample results in the generation of a cluster of immobilized amplicons on the internal surface of the reaction chamber at the predetermined location at which the targeting nucleic acid specific for that target polynucleotide was immobilized; and (d) detecting the position of clusters of amplicons on the solid surface to detect the presence of one or more target polynucleotides in the sample.

81-84. (canceled)
